SEQUENCE LISTING (17 sequences)

SEQ ID NO: 1 - DNA, 1887 bp, Zea mays
gtttcataaatgcttttcctgattccctcatcaattataaacctatataaggagtttgtggtataagcccgagatttgtccaatacccaaataacttcatctctcccttgagtgggggaatgctctccaagagcatataggagatcaccccaaatctgaaactcactcatagaaagagggcgagtaaaatcaataaaccactcgtcgtccacaaaacagtccgagaccaagacatccttgtccctacttatgtcaaagaaccttgggtacttgattttcaaaggaacatccccgagccatgtatccatccaaaatagggtgcacttcccattttcgatgttgtttattgcgccctatttaaataaatgtttaactttatgtaatccttttccaaaattgggaggtccttttcccattagataagaaaaaaaattgccatcaggcatatacttggctctaacaagtttgtaccaagtattatcagatcattgggagattttccatatccatgtaaccagaaggcactcattcatcggtctgctatctaaaaacaccaaacccccttgctccttcaacctagtcaccatttcccgtttagccataagataacagtttttcttttctgctccttgcgagaagaagtttgctctgatagagttcattttctggtgtgcctctttcgaaagaagatagaagctcatgttatacatcgggaggctactcagactggagttagtcaatatcagcctctccccagaggacagatacttaccctttcatgggtcaagcctcttattcatttttgctagaatagggtccgaaatagcagggttcagatgatggtgagaaatggccacacacctaggttgcggcgtttgtgcgggagctgttggagagggacttgctgatcacgttcggcgagggcaattaaggtatcgtagacatgttctacacggcagccatgtgcgggagcgtggacgtgttcactctactgctcaaccacgccacgaattgccggaacggccaaggcagcgcaaggcgtagtagctccatgttctgtacactgagataatgagtcgagtcgtccacgctgcagcgagaggcgggatcgtggagatgcatagggagttgctcgaggagagacagacgttcaggctattgctcgaccatgcatgtcaccgaggtgttcgacgaattgcaagtcacgctacactgccgactgagcaatacgagctactggcacccagcttgtgatttgaaagatgcacactaacaccaaacagcgaaacacccatggttcacgctcctcctaccacgtccacgacgaaaactgcatatgtagccacgtccacgtaggaccaaaacgagggacagaggaagcccatgcagcgttttcccgaaagacacgtaaagcagaacgtctccgctccgaggacgacacccgctcacgagcaatccggcagccagccgccgcaccgcagaatcttccccacgccacgctgccactgaaagcgcttcgacctcgtccgtccgttcgctcgctcgcggcgaaccccgcagagcttcccgtgcacgctcgcccgttccgttctgtgtggttggcagcctggcagcaccccacctgtccactcccctccactacgatacgagaccccggatccgtttttgctgtgtgctctaatcaaaaatcaaacaaaccagaagctcctcctcgcctcccatcacttcctacgccacccgcgaagcgcgcccgaggcggcaccccaccgtcgtagtagaagacacgggacgcacccccgcagcctcgctcgctcgctcccctcacttcctccccgcgcgatccacggcccccgccccccgcgctcctgtctgctctccctctccgca

SEQ ID NO: 2 - DNA, 1887 bp, Zea mays
gtttcataaatgcttttcctgattccctcatcaattataaacctatataaggagtttgtggtataagcccgagatttgtccaatacccaaataacttcatctctcccttgagtgggggaatgctctccaagagcatataggagatcaccccaaatctgaaactcactcatagaaagagggcgagtaaaatcaataaaccactcgtcgtccacaaaacagtccgagaccaagacatccttgtccctacttatgtcaaagaaccttgggtacttgattttcaaaggaacatccccgagccatgtatccatccaaaatagggtgcacttcccattttcgatgttgtttattgcgccctatttaaataaatgtttaactttatgtaatccttttccaaaattgggaggtccttttcccattagataagaaaaaaaattgccatcaggcatatacttggctctaacaagtttgtaccaagtattatcagatcattgggagattttccatatccatgtaaccagaaggcactcattcatcggtctgctatctaaaaacaccaaacccccttgctccttcaacctagtcaccatttcccgtttagccataagataacagtttttcttttctgctccttgcgagaagaagtttgctctgatagagttcattttctggtgtgcctctttcgaaagaagatagaagctcatgttatacatcgggaggctactcagactggagttagtcaatatcagcctctccccagaggacagatacttaccctttcatgggtcaagcctcttattcatttttgctagaatagggtccgaaatagcagggttcagatgatggtgagaaatggccacacacctaggttgcggcgtttgtgcgggagctgttggagagggacttgctgatcacgttcggcgagggcaattaaggtatcgtagacatgttctacacggcagccatgtgcgggagcgtggacgtgttcactctactgctcaaccacgccacgaattgccggaacggccaaggcagcgcaaggcgtagtagctccatgttctgtacactgagataatgagtcgagtcgtccacgctgcagcgagaggcgggatcgtggagatgcatagggagttgctcgaggagagacagacgttcaggctattgctcgaccatgcatgtcaccgaggtgttcgacgaattgcaagtcacgctacactgccgactgagcaatacgagctactggcacccagcttgtgatttgaaagatgcacactaacaccaaacagcgaaacacccatgtttcacgctcctcctaccacgtccacgacgaaaactgtatatgtagccacgtccacgtaggaccaaaacgagggacagaggaagcccatgcagcgttttcccgaaagacacgtaaagcagaacgtctccgctccgaggacgacacccgctcacgagcaatccggcagccagccgccgcaccgcagaatcttccccacgccacgctgccactgaaagcgcttcgacctcgtccgtccgttcgctcgctcgcggcgaaccccgcagagcttcccgtgcacgctcgcccgttccgttctgtgtggttggcagcctggcagcaccccacctgtccactcccctccactacgatacgagaccccggatccgtttttgctgtgtgctctaatcaaaaatcaaacaaaccagaagctcctcctcgcctcccatcacttcctacgccacccgcgaagcgcgcccgaggcggcaccccaccgtcgtagtagaagacacgggacgcacccccgcagcctcgctcgctcgctcccctcacttcctccccgcgcgatccacggcccccgccccccgcgctcctgtctgctctccctctccgca

SEQ ID NO: 3 - DNA, 2336 bp, Oryza sativa
gtaaatttacactagcaaaatgcccgtgcttcgctacgggtataatggaaggttaaatgctataaatacacggttaacatgtatgtaataatatttcaagatagataaattgttcattgggttaatgataatcaagactaataagaaacaccattgcatattgatatagtactcatattgtcataacacaagctctctctcaactcttgcatggcacgcgcatgatattacttctaaaatccaacacggattcgcgtggagacgaatcgcttaacagctcatgcgtggacgatagatggtgcggactctaatatgtcgtgttttgcagatgacgaaaagaaaaaaaaattatgtttaattttcaaattaaaaactggttcattataagatttcagaatcactaaatatctgttgaagaaagaggtataaaacttttgacttttcagccatcgaatgtgcatggtctgtgctaatggtggagagaaaaaaaaaggatgtgcatggtccgtgctagtggcggagagaaaaaaattgcacgcaaataggatttgagatatggagacaaagtagacttattccgagtaataataagtgtaaaagttttaggtagggtaggtcaatctggcctaggagaggcccatagacagtgcgagtaataatacaaaactcctaattagtgcatacgcgtcgcgaatttaagtggtgcgcgttttgagcccatccatcgcacagtcgcgtgcgacctattttcctttttttatattatcttttgtttcgagacttctttttcacgcagttggttgccacattgtcttctctattttttcgcacctcttgcgcaaggagtcggataaaaattgaaaaaaaaattcgcacagttggttgctaccgtgtctcctcttttctgttttcgcacctctttttttccttttacgtaattagtttccagtattaccatctactttacttcttctcttttttactaaaaattaaaaattactttaataattaaaaagttataaaaaaataagctataccaaaattgaatactttcacttaagatttcgaaacttcaactcgaatttcgaaaactttcaactacatatttgaaattttcaactattcttttaaaagttttaactaaattatttatgtgttttttctattgaactttgtttttagtgaattccctacctgttattatcctaacttaccgcataaaaaaggaaaaaaaaagataaagcgtcgggaggaatttttttcccaagaaaaaagcgaaaaaaaaacaaataaagaaaaatgaatcaataggtaggagagaaacttaaatgggccaaagcctgtaataataagagtaaaaaggtgagggtggcgggaacgaaccctggtggccacaatgaagattttcttcactaaccaagtaggcaagctgctacttgtgatcacatgttacatcaattaatacatatcctatattaacctagttttgaggtagatccaaaaggatccatcccgactattgagcggattaaaacaattttagcaacatgattctgattcggatgacggacgaaaaaaccatacggaaaccttacgaatttttttattaggtatagatgtcattataaacttttttaaaaaaattcaataatatagtttatgatgtaatatatcacttcacaaacccataacattgcatgatcaaatttaactttctacattttacaaaaaaaataaaaaaaaataattttgaatatacgttaattagttatagtttgcgaaagaacatgtattaacctgattagcgcctcaagatcgtacgttcaaatctccatttgaacgaattttagattgagttatttgagagctaaattttcaatttaaacaattatatatatccggttagatgtacatatcggataaataatacccttttttaaaaaaggattagttgtagtttaatttatttttccattgtgatttataaaaattgaatttgtgaagtgaaatgcaaatggcgaaatgacatttcttcgtttagcgtgaagcaacggagagaaggaacggaagcggtgggacgcgcgcgcacgccacgcgcgtagcgagagcgaaccagctcatccaccccgcgatccgtttttgctgtgccccaccgcctcagcgcctctccctcgcttaaaaccaaacccacacacccacctcttctctctctctcatcgtctccgcgactcagcccactcctctctctccaccaccaccaccaccaccaccaccgcccgccaagcgcggcccgcccgacacagcagcagcaggatcggcggagaggaggggggatc

SEQ ID NO: 4 - DNA, 809 bp, Oryza sativa
taaatggatttgaaggaagcagcgaatttctccgaggaccctcaatcttcctgtctttcatgagcggaatgaaaactttgtacactacatggaaagggaaccagttatgcaaagttgcaaacgatcactcaaggttacccttgtaggcctgctacttggccaatatggttccagtgaccatatgcagagtcaggttcagatgaatggcacttgtgagtagtgaagaataagatgaggatgcttgaagcggtttcacatgtggctgataccacgcaagcaacctctcaatgcatcgaaatgtgagtcttggaatcaataggatttagctcccatcaattacagttgtacccttttttgcttaatactttgtcgcctgtgctgttcttatatttgtgtgaagataaattttagtccattaggtaactgtattgttgagtcttaaggtgaagactaaatagtgtttggaagctgtagctactgcgatgtcaagtgtcaaaagagatcttggaaatcacaaggtgaagactaaatacttccttcgtttcacaatgtaagtcattctatggaaaatgctagaatgatttatattgtgaaatggagggagtagtgtttggaagcatgcggcatcagtcaggtgtgccttgtgttagcaggcacaggcactcgcatatgcagcacatttttccaccccttgtaatcaaatcaagttgctttatccaattatccttggcgtgatgccgtgatcacctaccccttgttatctcattaacctgctgtaagggcaatatggcctaatggcatagagaagg

SEQ ID NO: 5 - DNA, 1009 bp, Oryza sativa
taaatggatttgaaggaagcagcgaatttctccgaggaccctcaatcttcctgtctttcatgagcggaatgaaaactttgtacactacatggaaagggaaccagttatgcaaagttgcaaacgatcactcaaggttacccttgtaggcctgctacttggccaatatggttccagtgaccatatgcagagtcaggttcagatgaatggcacttgtgagtagtgaagaataagatgaggatgcttgaagcggtttcacatgtggctgataccacgcaagcaacctctcaatgcatcgaaatgtgagtcttggaatcaataggatttagctcccatcaattacagttgtacccttttttgcttaatactttgtcgcctgtgctgttcttatatttgtgtgaagataaattttagtccattaggtaactgtattgttgagtcttaaggtgaagactaaatagtgtttggaagctgtagctactgcgatgtcaagtgtcaaaagagatcttggaaatcacaaggtgaagactaaatacttccttcgtttcacaatgtaagtcattctatggaaaatgctagaatgatttatattgtgaaatggagggagtagtgtttggaagcatgcggcatcagtcaggtgtgccttgtgttagcaggcacaggcactcgcatatgcagcacatttttccaccccttgtaatcaaatcaagttgctttatccaattatccttggcgtgatgccgtgatcacctaccccttgttatctcattaacctgctgtaagggcaatatggcctaatggcatagagaagagcctccagtgctccatacctagacgtccaggcatctcattaaggtatcaatccttcatctttgtgagtgaccaacccagattcccgtccttcgaaaaagagagagaaatgattcatgagccatcaacggatcgaccgcaagatagaaacaaatactacatctgtacatcagtccgtcgcgcaatataaggtatttgacag

SEQ ID NO: 6 - DNA, 15 bp, Artificial Sequence
  primer sequence
ntcgastwtsgwgtt

SEQ ID NO: 7 - DNA, 16 bp, Artificial Sequence
  primer sequence
ngtcgaswganawgaa

SEQ ID NO: 8 - DNA, 16 bp, Artificial Sequence
  primer sequence
wgtgnagwancanaga

SEQ ID NO: 9 - DNA, 27 bp, Artificial Sequence
  primer sequence
gccgtgtagaacatgtctacgatacct

SEQ ID NO: 10 - DNA, 30 bp, Artificial Sequence
  primer sequence
tttcatatgtgcggagagggagagcagaca

SEQ ID NO: 11 - DNA, 37 bp, Artificial Sequence
  primer sequence
tttaagcttgtttcataaatgcttttcctgattccct

SEQ ID NO: 12 - DNA, 26 bp, Artificial Sequence
  primer sequence
ccgtcgccatggatcccccctcctct

SEQ ID NO: 13 - DNA, 35 bp, Artificial Sequence
  primer sequence
ccaagcttgtaaatttacactagcaaaatgcccgt

SEQ ID NO: 14 - DNA, 31 bp, Artificial Sequence
  primer sequence
aaccatggatcccccctcctctccgccgatc

SEQ ID NO: 15 - DNA, 30 bp, Artificial Sequence
  primer sequence
attaccgcggtaaatggatttgaaggaagc

SEQ ID NO: 16 - DNA, 30 bp, Artificial Sequence
  primer sequence
atatggatcccttctctatgccattaggcc

SEQ ID NO: 17 - DNA, 29 bp, Artificial Sequence
  primer sequence
ataggatcctgtcaaataccttatattgc

(19) United States
(12) Patent Application Publication
(10) Pub. No.: US 2010/0043100 A1
(43) Pub. Date: Feb. 18, 2010

(54) POLYNUCLEOTIDES FOR REGULATION OF HIGH LEVEL TISSUE-PREFERRED EXPRESSION IN CROP PLANTS

(75) Inventors: Christopher Kafer, Raleigh, NC (US); Hanping Guan, Chapel Hill, NC (US)

Correspondence Address: CONNOLLY BOVE LODGE & HUTZ, LLP, P O BOX 2207, WILMINGTON, DE, 19899, US

(73) Assignee: BASF PLANT SCIENCE GMBH, Ludwigshafen (DE)

(21) Appl. No.: 12/595,276

(22) PCT Filed: Apr. 7, 2008
(86) PCT No.: PCT/EP2008/054130

§ 371 Date: Oct. 9, 2009

Related U.S. Application Data
(60) Provisional application No. 60/911,537, filed on Apr. 13, 2007.

(51) Int. Cl. A01H 1/00; C07H 21/04; A01H 5/00; C12N 15/63; C12N 5/10

(52) U.S. Cl. 800/278; 536/23.6; 800/298; 435/320.1; 435/419

(57) ABSTRACT

This invention provides polynucleotides regulating high level tissue-preferred expression. Compositions comprising the polynucleotides include DNA constructs useful for plant transformation and plants transformed with such DNA constructs. Further provided are methods for the expression of transgenes in plants using the tissue-preferred regulatory elements.

### FIELD OF THE INVENTION

The present invention relates to the field of agricultural biotechnology. Disclosed herein are isolated nucleic acids capable of directing high level tissue-preferred expression in crop plants, expression vectors containing the same, and plants generated thereof.

### BACKGROUND OF THE INVENTION

In grain crops of agronomic importance, seed formation is the ultimate goal of plant development. Seeds are harvested for use in food, feed, and industrial products. The utility and value of those seeds are determined by the quantity and quality of protein, oil, and starch contained therein. In turn, the quality and quantity of seed produced may be affected by environmental conditions at any point prior to fertilization through seed maturation. In particular, stress at or around the time of fertilization may have substantial impact on seed development. Members of the grass family, which include the cereal grains, produce dry and one-seeded fruits. This type of fruit is, strictly speaking, a caryopsis but is commonly called a kernel or grain. A kernel or grain comprises a seed and its coat or pericarp. A seed comprises an embryo or germ, an endosperm enclosed by a nucellar epidermis and a seed coat. An embryo is the miniature progenitor of the next generation, containing cells for root and shoot growth of a new plant. It is also the tissue in which oil and proteins are stored in a kernel. An endosperm functions more as a nutritive tissue and provides the energy needed in the form of stored starch, proteins and oil needed for the germination and initial growth of the embryo.

Considering the complex regulation that occurs during kernel development in higher plants, and that the grain is the common primary source of nutrition for animals and humans, key tools needed to improve such nutrition source include genetic promoters that can drive the expression of genes enhancing nutrition. On the other hand, kernels are sensitive toward stresses. Stresses to plants may be caused by both biotic and abiotic agents. For example, biotic causes of stress include infection with a pathogen, insect feeding, and parasitism by another plant such as mistletoe, and brazing by ruminant animals. Abiotic stresses include, for example, excessive or insufficient available water, insufficient light, temperature extremes, synthetic chemicals such as herbicides, excessive wind, extreme soil pH, limited nutrient availability, and air pollution. Yet plants survive and often flourish, even under unfavorable conditions, using a variety of internal and external mechanisms for avoiding or tolerating stress. Plants' physiological responses to stresses reflect changes in gene expression.

While manipulation of stress-induced genes may play an important role in improving plant tolerance to stresses, it has been shown that constitutive expression of stress-inducible genes has a severe negative impact on plant growth and development when the stress is not present. Therefore, there is a need in the art for promoters driving expression which is temporally- and/or spatially-differentiated, to provide a means to control and direct gene expression in specific cells or tissues at critical times, especially to provide stress tolerance or avoidance. In particular, drought and/or density stress of maize often results in reduced yield. To stabilize plant development and grain yield under unfavorable environments, manipulation of hormones and nutritional supply to kernel during seed germination is of interest. Thus there is a need for transcription regulatory elements which drive gene expression in endosperm, embryo, or kernel under normal or abiotic stress conditions.

Promoters that confer enhanced expression during seed or grain maturation are described, such as the barley hordein promoters in US patent application 20040088754. Promoters that direct embryo-specific or seed-specific expression in dicots (e.g., the soybean conglycinin promoter, Chen 1988; the napin promoter, Kridl 1991) are generally not capable to direct similar expression in monocots. Unfortunately, relatively few promoters specifically directing to this aspect of physiology have been identified (see for example US20040163144).

Accordingly there is a need in the art for regulatory sequences which allow for expression in kernel, embryo, or endosperm during seed development. Seed- or grain-specific promoters described include those associated with genes that encode plant seed storage proteins such as genes encoding: barley hordeins; rice glutelins, oryzins, prolamines, or globulins; wheat gliadins or glutenins; maize zeins or glutelins; oat glutelins; sorghum kafirins; millet pennisetins; or rye secalins. However, expression of these promoters is often leaky or of low expression level. Furthermore, improvement of crop plants with multiple transgenes (“stacking”) is of increasing interest. For example, a single maize hybrid may contain recombinant DNA conferring not only insect resistance, but also resistance to a specific herbicide. However, the phenomena of gene silencing is often observed when identical regulatory sequences are used to drive expression of multiple genes. Metabolic engineering of crops may require multiple novel regulatory DNA sequences driving expression of different transgenes within the same plant in a tissue or temporally specific pattern.

There is, therefore, a great need in the art for the identification of novel sequences that can be used for expression of selected transgenes in economically important plants. There is also a need in the art for transcription regulating sequences that allow for expression in endosperm or kernel during seed development. It is thus an objective of the present invention to provide new and alternative regulatory sequences for endosperm-preferred or kernel-preferred expression. The objective is solved by the present invention.

### SUMMARY OF THE INVENTION

The present invention relates to the field of agricultural biotechnology. Disclosed herein are isolated nucleic acids capable of directing high level tissue-preferred expression in crop plants, expression vectors containing the same, and plants generated thereof. Therefore, the first embodiment of the invention relates to an isolated plant transcription regulatory element comprising a polynucleotide selected from the group consisting of: a) a polynucleotide having a sequence as defined in SEQ ID NO:1, 2, or 3; b) a polynucleotide having 70% sequence identity to a polynucleotide having a sequence as defined in SEQ ID NO:1, 2, or 3; c) a polynucleotide having a fragment of at least 50 consecutive nucleotides, or at least 100 consecutive nucleotides, or at least 200 consecutive nucleotides of a polynucleotide having a sequence as defined in SEQ ID NO:1, 2, or 3; d) polynucleotide hybridizing under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:1, 2, or 3; and e) a polynucleotide complementary to any of the polynucleotides of a) through d).

In one embodiment, the plant transcription regulatory element regulates endosperm-preferred expression of a gene of interest in a plant or plant cell. In another embodiment, the plant or plant cell is a monocot or a dicot. One of skill in the art would recognize that this plant or plant cell could be, but not be limited to, maize, wheat, rice, barley, oat, rye, sorghum, banana, ryegrass, pea, alfalfa, soybean, carrot, celery, tomato, potato, cotton, tobacco, pepper, oilseed rape, beet, cabbage, cauliflower, broccoli, lettuce and Arabidopsis thaliana. Further, one of skill in the art would recognize that a plant transcription regulatory element could be operably linked to one or more nucleic acids.

Yet another embodiment relates to a plant transformed with an isolated plant transcription regulatory element comprising a polynucleotide selected from the group consisting of:
a) a polynucleotide having a sequence as defined in SEQ ID NO:1, 2, or 3;
b) a polynucleotide having 70% sequence identity to a polynucleotide having a sequence as defined in SEQ ID NO:1, 2, or 3;
c) a polynucleotide having a fragment of at least 50 consecutive nucleotides, or at least 100 consecutive nucleotides, or at least 200 consecutive nucleotides of a polynucleotide having a sequence as defined in SEQ ID NO:1, 2, or 3;
d) a polynucleotide hybridizing under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:1, 2, or 3; and
e) a polynucleotide complementary to any of the polynucleotides of a) through d).

In one embodiment, the plant transcription regulatory element is operably linked one or more heterologous nucleic acids. These heterologous nucleic acids encode polypeptides that confer to a plant a trait or property selected from the group consisting of increased yield, increased resistance under stress conditions, increased nutritional quality, increased or modified starch content, and/or increased or modified oil content of a seed or sprout. The increased nutritional quality and/or oil content may comprise an increased content of at least one compound selected from the group consisting of vitamins, carotinoids antioxidants, unsaturated fatty acid, polyunsaturated fatty acids, and proteins with altered amino acid content. It is also preferred that the transcription of the functionally linked nucleic acid in the expression vector results in the expression of a protein or expression of a functionally ribonucleotide capable to impact function of at least one gene in the target plant. The functional RNA comprises at least antisense RNA, sense RNA, dsRNA, microRNA, siRNA, or combination thereof.

In another embodiment, the operably linked heterologous nucleic acid expresses regulatory RNA selected from the group consisting of dsNA, siRNA, and miRNA. In yet another embodiment, the transcription regulatory element regulates endosperm-preferred expression of one or more operably linked nucleic acids. In another embodiment, the plant is a monocot or a dicot. One of skill in the art would recognize that the plant could be, but not be limited to, maize, wheat, rice, barley, oat, rye, sorghum, banana, ryegrass, pea, alfalfa, soybean, carrot, celery, tomato, potato, cotton, tobacco, pepper, oilseed rape, beet, cabbage, cauliflower, broccoli, lettuce and Arabidopsis thaliana.

Other embodiments of the invention relate to a nucleic acid construct or an expression vector comprising the plant transcription regulatory sequence operably linked to one or more heterologous nucleic acids.

One embodiment of the invention provides a seed produced by a transgenic plant transformed by the transcription regulatory element operably linked to one or more nucleic acids. The seed produced by the transgenic plant expresses a protein or a functional RNA capable of impacting function of at least one gene in the target plant, wherein the seed or plant has increased resistance under stress conditions, and/or increased yield, and/or increased nutritional quality, and/or increased or modified starch content, and/or increased or modified oil content of a seed or a sprout. In another embodiment, the seed or plant is a monocot or a dicot. In yet another embodiment, the seed or plant is selected from the group consisting of maize, wheat, rice, barley, oat, rye, sorghum, banana, and ryegrass.

Another embodiment of the invention relates to a method for increased yield, increased stress tolerance, increased nutritional quality, increased nutritional value, increased or modified starch content, and/or increased or modified oil content of a seed or a sprout of a plant, wherein the method comprises the steps of:
1) introducing into a plant cell or a plant an expression vector comprising:
   A) a plant transcription regulatory element selected from the group consisting of:
      a) a polynucleotide having a sequence as defined in SEQ ID NO:1, 2, or 3;
      b) a polynucleotide having 70% sequence identity to a polynucleotide having a sequence as defined in SEQ ID NO:1, 2, or 3;
      c) a polynucleotide having a fragment of at least 50 consecutive nucleotides, or at least 100 consecutive nucleotides, or at least 200 consecutive nucleotides of a polynucleotide having a sequence as defined in SEQ ID NO:1, 2, or 3;
      d) a polynucleotide hybridizing under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:1, 2, or 3; and
      e) a polynucleotide complementary to any of the polynucleotides of a) through d); operably linked to one or more heterologous nucleic acids;
   B) wherein the operably linked nucleic acid encodes a polypeptide or RNA that is capable of conferring to a plant increased yield, increased stress tolerance, increased nutritional quality, increased nutritional value, increased or modified starch content, or increased or modified oil content to the plant;
2) selecting transgenic plants, wherein the plants have increased yield, increased stress tolerance under stress conditions, increased nutritional quality, increased nutritional value, increased or modified starch content, or increased or modified oil content of a seed or a sprout of the plants, as compared to the wild type or null segregant plants.

Various nucleic acids are known to the person skilled in the art to obtain yield and/or stress resistance. The nucleic acids may include, but are not limited to polynucleotides encoding a polypeptide involved in phytohormone biosynthesis, phytohormone regulation, cell cycle regulation, or carbohydrate metabolism. The nutritional quality, nutritional value, starch content and oil content are defined as below.

Another embodiment of the invention relates to an isolated plant transcription terminator element comprising a polynucleotide selected from the group consisting of:
a. a polynucleotide having a sequence as defined in SEQ ID NO:4 or SEQ ID NO:5;
b. a polynucleotide having 70% sequence identity to a polynucleotide having a sequence as defined in SEQ ID NO:4 or SEQ ID NO:5;
c. a polynucleotide having a fragment of at least 50 consecutive nucleotides, or at least 100 consecutive nucleotides, or at least 200 consecutive nucleotides of a polynucleotide having a sequence as defined in SEQ ID NO:4 or SEQ ID NO:5;
d. a polynucleotide hybridizing under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:4 or SEQ ID NO:5; and
e. a polynucleotide complementary to any of the polynucleotides of a) through d).

### BRIEF DESCRIPTION OF THE DRAWING

FIG. 1 sets forth the Maize SSI promoter region (pEXS1033) (SEQ ID NO:1).

FIG. 2 sets forth the Maize SSI promoter (mutNcoINdeI) region from plasmid pEXS1032: (SEQ ID NO:2)

FIG. 3 sets forth the OsSSI promoter (pEXS1031) (SEQ ID NO:3).

FIG. 4 sets forth the OsSSI terminator from plasmid RLM661 (t-OSSSI-3) (SEQ ID NO:4).

FIG. 5 sets forth the OsSSI terminator from plasmid RLM662 (t-OSSSI-5) (SEQ ID NO:5).

FIGS. 6a and 6b show the sequence alignment of SEQ ID NO:1 and SEQ ID NO:2. The analysis was performed in Vector NTI software suite using the Fast Algorithm (gap opening 15, gap extension 6.66 and gap separation 8, matrix is swgapdnamt).

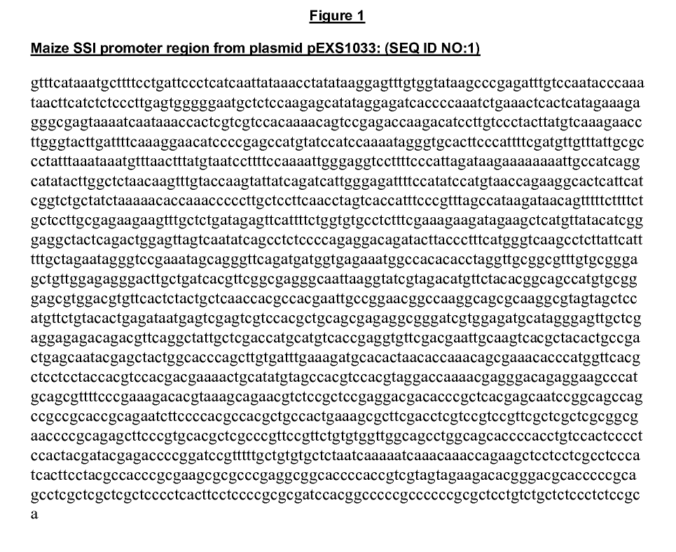

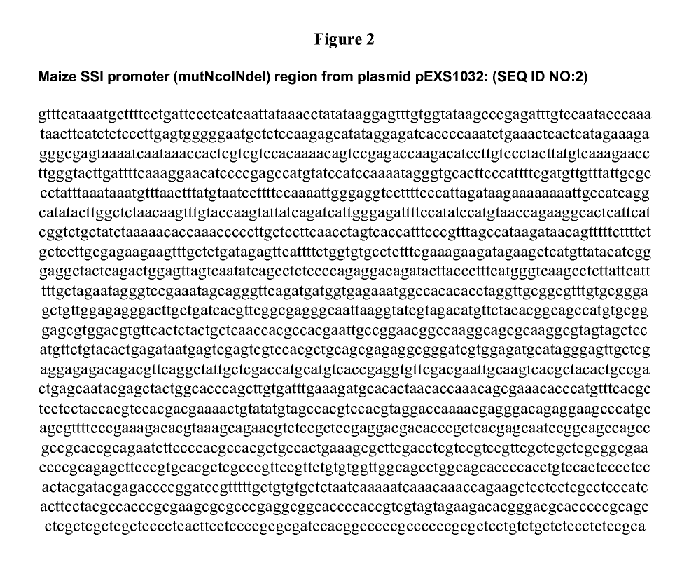

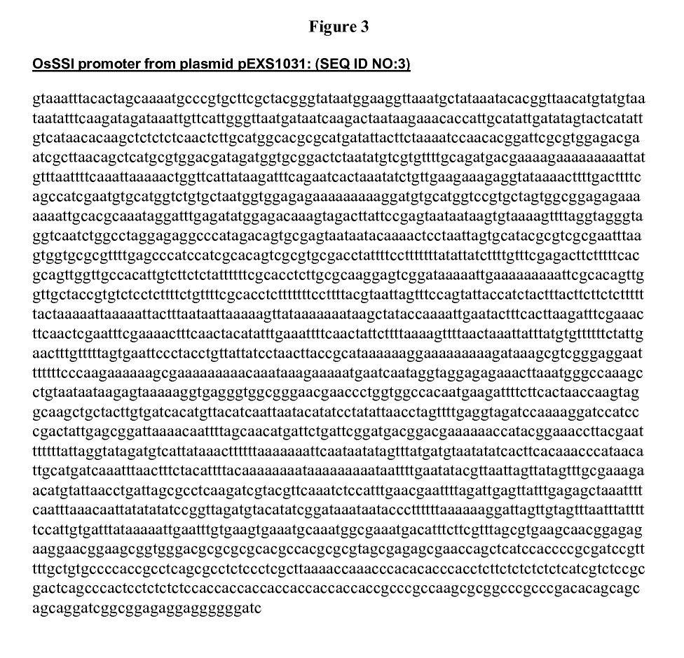

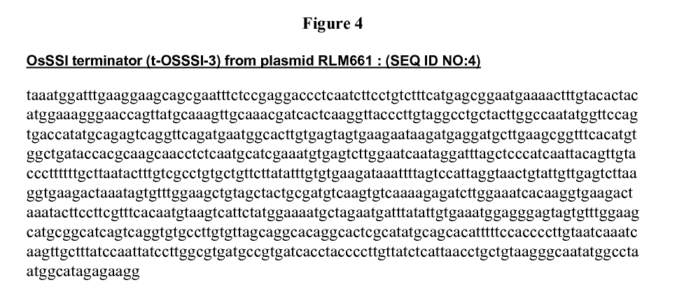

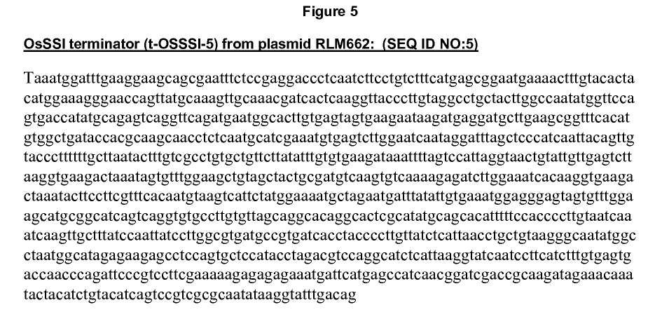

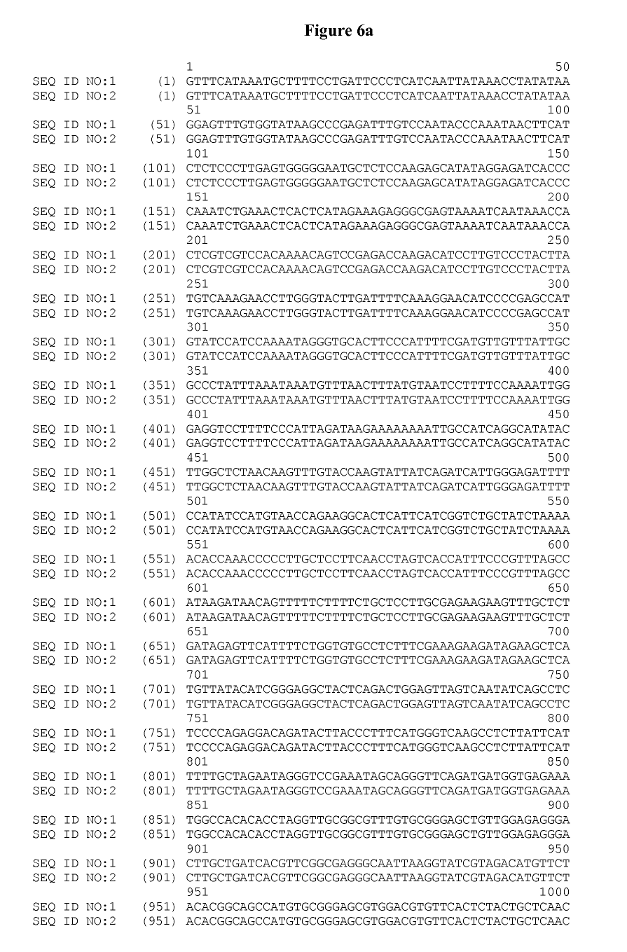

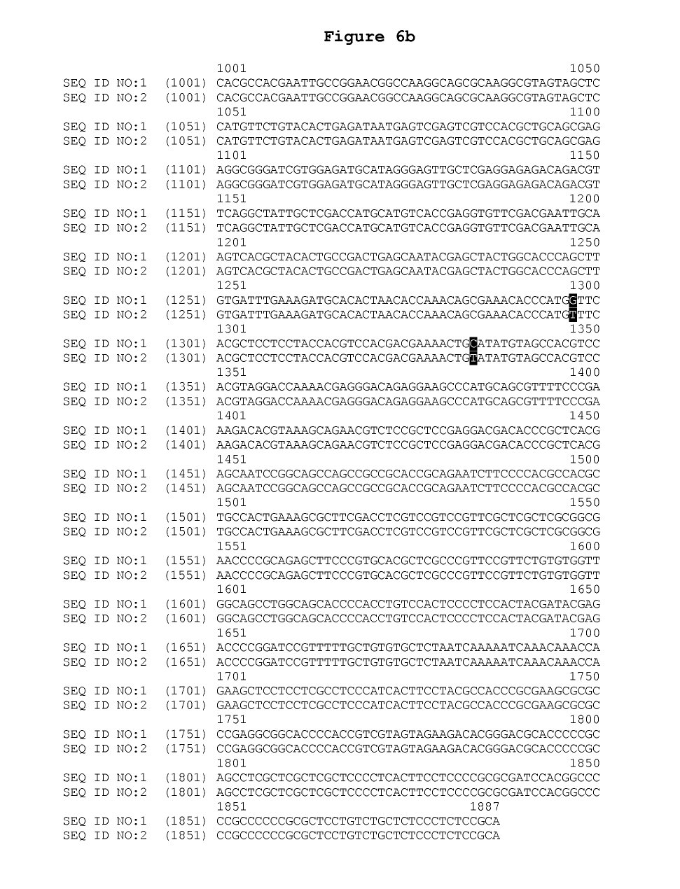

### DETAILED DESCRIPTION OF THE INVENTION

Unless otherwise noted, the terms used herein are to be understood according to conventional usage by those of ordinary skilled in the relevant art. Definition of common terms in molecular biology may be found in many reference sources known to those of skill in the art, including but not limited to, Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc., and John Wiley & Sons, Inc., (1998 Supplement).

It must be noted that as used herein and in the appended claims, the singular form “a”, “an”, or “the” includes plural reference unless the context clearly dictates otherwise.

As used herein, the word “or” means any one member of a particular list and also includes any combination of members of that list.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

As used herein, the word “nucleic acid”, “nucleotide”, or “polynucleotide” is intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. A polynucleotide may encode for an agronomically valuable or phenotypic trait.

The term “gene” is used broadly to refer to any segment of nucleic acid associated with a biological function. Thus, genes include introns and exons as in genomic sequence, or just the coding sequences as in cDNAs and/or the regulatory sequences required for their expression. For example, a gene refers to a nucleic acid fragment that expresses mRNA or functional RNA, or encodes a specific protein, and which includes regulatory sequences.

The terms “polypeptide” and “protein” are used interchangeably herein to refer to a polymer of consecutive amino acid residues.

The term “transgene” as used herein refers to any polynucleotide that is introduced into the genome of a cell by experimental manipulations. A transgene may be an “endogenous DNA”, or a “heterologous DNA”. “Endogenous DNA” refers to a polynucleotide that is naturally found in the cell into which it is introduced so long as it does not contain any modification relative to the naturally occurring polynucleotide. “Heterologous DNA” refers to a polynucleotide that is ligated to a polynucleotide to which it is not ligated in nature, or to which it is ligated at a different location in nature. Heterologous DNA is not endogenous to the cell into which it is introduced, but has been obtained from another cell. Heterologous DNA can include an endogenous DNA that contains some modification.

The term “cell” or “plant cell” as used herein refers to single cell, and also includes a population of cells. The population may be a pure population comprising one cell type. Likewise, the population may comprise more than one cell type. A plant cell within the meaning of the invention may be isolated (e.g., in suspension culture) or comprised in a plant tissue, plant organ or plant at any developmental stage.

The term “operably linked” or “functionally linked” as used herein refers to the association of nucleic acid sequences so that the function of one is affected by the other. For example, a regulatory DNA is said to be “operably linked” to a DNA that expresses an RNA or encodes a polypeptide if the two DNAs are situated such that the regulatory DNA affects the expression of the coding DNA.

The term “specific” or “preferred” expression as used herein refers to the expression of gene products that is limited primarily to one or a few plant tissues (spacial limitation) and/or to one or a few plant developmental stages (temporal limitation). It is acknowledged that a true specificity rarely exists: promoters seem to be preferably switched on in some tissues, while in other tissues there can be no or only little activity. This phenomenon may also be referred to as leaky expression with varying levels of “leakiness”. Leakiness can also be exhibited as preferred expression in one or a few plant tissues with a lower level of constitutive expression elsewhere in the plant.

The term “5′ non-coding region” or “5′untranslated region” or “5′UTR” as used herein refers to a nucleotide sequence located 5′ (upstream) to a coding sequence. It is present in the fully processed mRNA upstream of the initiation codon and may affect processing of the primary transcript, the mRNA stability, or translation efficiency.

The term “3′ non-coding region” or “3′untranslated region” or “3′UTR” as used herein refers to nucleotide sequence located 3′ (downstream) to a coding sequence, and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “transcription regulatory element” as used herein refers to a polynucleotide that is capable of regulating the transcription of an operably linked polynucleotide. It includes, but not limited to, promoters, enhancers, introns, 5′UTRs, 3′UTRs, and polyadenylation sequences.

As used herein, “RNAi” or “RNA interference” refers to the process of sequence-specific post-transcriptional gene silencing, mediated by double-stranded RNA (dsRNA). As used herein, “dsRNA” refers to RNA that is partially or completely double stranded. Double stranded RNA is also referred to as small or short interfering RNA (siRNA), short interfering nucleic acid (siNA), short interfering RNA, micro-RNA (miRNA), and the like. In the RNAi process, dsRNA comprising a first strand that is substantially identical to a portion of a target gene and a second strand that is complementary to the first strand is introduced into a host cell. After the introduction, the target gene-specific dsRNA is processed into relatively small fragments (siRNAs) and can subsequently become distributed throughout the host cell, leading to a loss-of-function mutation having a phenotype that, over the period of a generation, may come to closely resemble the phenotype arising from a complete or partial deletion of the target gene. Alternatively, the target gene-specific dsRNA is processed into relatively small fragments of about 19-24 bp by a plant cell containing the RNAi processing machinery.

The term “cell” or “plant cell” as used herein refers to single cell, and also includes a population of cells. The population may be a pure population comprising one cell type. Likewise, the population may comprise more than one cell type. A plant cell within the meaning of the invention may be isolated (e.g., in suspension culture) or comprised in a plant tissue, plant organ or plant at any developmental stage.

The term “tissue” with respect to a plant (or “plant tissue”) means arrangement of multiple plant cells, including differentiated and undifferentiated tissues of plants. Plant tissues may constitute part of a plant organ (e.g., the epidermis of a plant leap but may also constitute tumor tissues (e.g., callus tissue) and various types of cells in culture (e.g., single cells, protoplasts, embryos, calli, protocorm-like bodies, etc.). Plant tissues may be in planta, in organ culture, tissue culture, or cell culture.

The term “organ” with respect to a plant (or “plant organ”) means parts of a plant and may include, but not limited to, for example roots, fruits, shoots, stems, leaves, hypocotyls, cotyledons, anthers, sepals, petals, pollen, seeds, etc.

The term “plant” as used herein can, depending on context, be understood to refer to whole plants, plant cells, plant organs, sprouts, plant seeds, and progeny of same. The word “plant” also refers to any plant, particularly, to seed plant, and may include, but not limited to, crop plants. The class of plants is generally as broad as the class of higher and lower plants amenable to transformation techniques, including angiosperms (monocotyledonous and dicotyledonous plants), gymnosperms, ferns, horsetails, psilophytes, bryophytes, and multicellular algae. The plant can be from a genus selected from the group consisting of Medicago, Lycopersicon, Brassica, Cucumis, Solanum, Juglans, Gossypium, Malus, Vitis, Antirrhinum, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Medicago, Onobrychis, trifolium, Trigonella, Vigna, Citrus, Linum, Geranium, Manihot, Daucus, Raphanus, Sinapis, Atropa, Datura, Hyoscyamus, Nicotiana, Petunia, Digitalis, Majorana, Ciahorium, Lactuca, Bromus, Asparagus, Antirrhinum, Heterocallis, Nemesis, Pelargonium, Panieum, Pennisetum, Ranunculus, Senecio, Salpiglossis, Browaalia, Phaseolus, Avena, and Allium.

The term “plant” as used herein can be monocotyledonous crop plants, such as, for example, cereals including wheat, barley, sorghum, rye, triticale, maize, rice, sugarcane, and trees including apple, pear, quince, plum, cherry, peach, nectarine, apricot, papaya, mango, poplar, pine, sequoia, cedar, and oak. The term “plant” as used herein can be dicotyledonous crop plants, such as pea, alfalfa, soybean, carrot, celery, tomato, potato, cotton, tobacco, pepper, oilseed rape, beet, cabbage, cauliflower, broccoli, lettuce and Arabidopsis thaliana.

The term “transgenic” as used herein is intended to refer to cells and/or plants which contain a transgene, or whose genome has been altered by the introduction of a transgene, or that have incorporated exogenous genes or polynucleotides. Transgenic cells, tissues, organs and plants may be produced by several methods including the introduction of a “transgene” comprising polynucleotide (usually DNA) into a target cell or integration of the transgene into a chromosome of a target cell by way of human intervention, such as by the methods described herein.

The term “true breeding” as used herein refers to a variety of plant for a particular trait if it is genetically homozygous for that trait to the extent that, when the true-breeding variety is self-pollinated, a significant amount of independent segregation of the trait among the progeny is not observed.

The term “null segregant” as used herein refers to a progeny (or lines derived from the progeny) of a transgenic plant that does not contain the transgene due to Mendelian segregation.

The term “wild type” as used herein refers to a plant cell, seed, plant component, plant tissue, plant organ, or whole plant that has not been genetically modified or treated in an experimental sense.

The term “control plant” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein.

The term “trait” as used herein refers to a physiological, morphological, biochemical, or physical characteristic of a plant or particular plant material or cell. In some instances, this characteristic is visible to human eyes, such as seed or plant size, or can be measured by biochemical techniques, such as detecting the protein, starch, or oil content of seed or leaves, or by observation of a metabolic or physiological process, for example, by measuring tolerance to water deprivation or particular salt or sugar concentrations, or by the observation of the expression level of a gene or genes, e.g., by employing Northern analysis, RT-PCT, microarray gene expression assays, or by agricultural observations such as osmotic stress tolerance or yield. Any technique can be used to measure the amount of, comparative level of, or difference in any selected chemical compound or macromolecule in the transgenic plants.

The present invention may be understood more readily by reference to the following detailed description of the preferred embodiments of the invention and the Examples included herein. However, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting.

Standard techniques for cloning, DNA and RNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook and Russell, 2001 Molecular Cloning, Third Edition, Cold Spring Harbor Laboratory, Plainview, N.Y.; Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

The present invention describes for the first time the regulatory element upstream of a starch synthase gene. Accordingly, one embodiment of the invention relates to an isolated plant transcription regulatory element comprising a polynucleotide selected from the group consisting of:
a) a polynucleotide having a sequence as defined in SEQ ID NO:1, 2, or 3;
b) a polynucleotide having 70% sequence identity to a polynucleotide having a sequence as defined in SEQ ID NO:1, 2, or 3;
c) a polynucleotide having a fragment of at least 50 consecutive nucleotides, or at least 100 consecutive nucleotides, or at least 200 consecutive nucleotides of a polynucleotide having a sequence as defined in SEQ ID NO:1, 2, or 3;
d) a polynucleotide hybridizing under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:1, 2, or 3; and
e) a polynucleotide complementary to any of the polynucleotides of a) through d).

The present invention also embodies an isolated plant transcription regulatory element comprising a polynucleotide sequence which is at least about 50-60%, or at least about 60-70%, or at least about 70-80%, 80-85%, 85-90% or 90-95%, or at least about 95%, 96%, 97%, 98%, 99% or more identical or similar to a nucleotide sequence as defined in SEQ ID NO:1, SEQ ID NO:2, or SEQ ID NO:3, or a portion thereof. The length of the sequence comparison for nucleic acids is at least 50 consecutive nucleotides, or at least 100 consecutive nucleotides, or at least 200 consecutive nucleotides. The sequence identity and sequence similarity are defined as below.

In another embodiment, an isolated nucleic acid of the invention hybridizes under stringent conditions to a polynucleotide as defined in SEQ ID NO:1, SEQ ID NO:2, or SEQ ID NO:3, or a portion thereof. In another embodiment, an isolated nucleic acid hybridizes under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:1, 2, or 3, or a portion thereof. As used herein, the term “hybridizes under stringent conditions” is intended to describe conditions for hybridization and washing under which nucleotides at least 60% similar or identical to each other typically remain hybridized to each other. In another embodiment, the conditions are such that nucleotides at least about 65%, or at least about 70%, or at least about 75%, or at least about 80% or more similar or identical to each other typically remain hybridized to each other. Such stringent conditions are known to those skilled in the art and described as below. A preferred, non-limiting example of stringent conditions are hybridization in 6× sodium chloride/sodium citrate (SSC) at about 45° C., followed by one or more washes in 0.2×SSC, 0.1% SDS at 50-65° C.

“Hybridization” can be used to indicate the level of similarity or identity between two nucleic acid molecules, and also to detect the presence of the same or similar nucleic acid molecule in Southern or Northern analyses. Hybridization of two DNA molecules is dependent upon the parameters of both hybridization procedure and the wash conditions. “Stringent hybridization conditions” and “stringent hybridization wash conditions” are sequence dependent, and are different under different environmental parameters. The stability of the hybrid is expressed as the melting temperature Tm that is the temperature at which 50% of the target sequence hybridizes to a perfectly matched probe. Specificity is typically the function of post-hybridization washes, the critical factors being the ionic strength and temperature of the final wash solution. For DNA-DNA hybridization, the Tm can be approximated by the following equation (Sambrook et. al., 1989):

Tm=81.5° C.+16.6(log₁₀[Na⁺]+0.41(% G+C)−0.61(% formamide)−(600/L) a)

Where: Na⁺=molarity of monovalent sodium cations
G+C=percentage of guanosine and cytosine nucleotides in the DNA
L=length of the hybrid in base pairs
   For hybridization between two heterologous sequences, the Tm of the double-stranded hybrid decreases by about 1° C. with every 1% decrease in homology (Sambrook et. al., 1989). Thus Tm, hybridization, and/or wash conditions can be adjusted for hybridization of sequences with desired identity. Generally, stringent conditions are selected to be about 5° C. lower than the Tm for the specific sequence at a defined ionic strength and pH. However, severely stringent conditions can utilize a hybridization and/or wash at 1, 2, 3, or 4° C. lower than the Tm; moderately stringent conditions can utilize a hybridization and/or wash at 6, 7, 8, 9, or 10° C. lower than the Tm; low stringency conditions can utilize a hybridization and/or wash at 11, 12, 13, 14, 15, or 20° C. lower than the Tm. Using the equation, hybridization and wash compositions, and desired T, those of ordinary skill will understand that variations in the stringency of hybridization and/or wash solutions are inherently described. A preferred, non-limiting example of stringent hybridization conditions are described above.

An “antisense” nucleic acid comprises a nucleotide sequence that is complementary to a “sense” nucleic acid encoding a protein, e.g., complementary to an mRNA sequence. Accordingly, an antisense nucleic acid can hydrogen bond to a sense nucleic acid.

The antisense nucleic acid can be complementary to an entire target polynucleotide, or to a portion thereof. The antisense nucleic acid molecules are typically administered to a cell or generated in situ such that they hybridize with or bind to cellular mRNA and/or genomic DNA. Hybridization may be performed under stringent conditions as described above.

In yet another embodiment, an isolated nucleic acid is complementary to a polynucleotide as defined in SEQ ID NO:1 or SEQ ID NO:2, or to a polynucleotide having 70% sequence identity to a polynucleotide as defined in SEQ ID NO:1 or SEQ ID NO:2, or to a polynucleotide hybridizing to the polynucleotide as defined in SEQ ID NO:1. As used herein, “complementary” polynucleotides refer to those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA.

The present invention further encompasses the regulatory element upstream of a starch synthase derived from maize or rice that is capable of regulating tissue-specific expression of an operably linked heterologous nucleic acid in crop plants. Therefore another embodiment of the invention relates to an isolated plant transcription regulatory element comprising a polynucleotide, wherein the polynucleotide is selected from the group consisting of: a) a polynucleotide having a sequence as defined in SEQ ID NO:1, 2, or 3; b) a polynucleotide having 70% sequence identity to a polynucleotide having a sequence as defined in SEQ ID NO:1, 2, or 3; c) a polynucleotide having a fragment of at least 50 consecutive nucleotides, or at least 100 consecutive nucleotides, or at least 200 consecutive nucleotides of a polynucleotide having a sequence as defined in SEQ ID NO:1, 2, or 3; d) a polynucleotide hybridizing under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:1, 2, or 3; and e) a polynucleotide complementary to any of the polynucleotides of a) through d); and wherein the plant transcription regulatory element regulates endosperm-specific expression of a heterologous nucleic acid of interest in a plant or plant cell.

In one embodiment, an isolated nucleic acid as specified under a), b), c), d), and e) of any of the transcription regulatory elements is capable of modifying transcription in a plant, preferably it is capable of regulating endosperm-preferred expression in a plant. In another embodiment, an isolated nucleic acid of the invention comprises a nucleotide sequence which is at least about 50-60%, or at least about 60-70%, or at least about 70-80%, 80-85%, 85-90% or 90-95%, or at least about 95%, 96%, 97%, 98%, 99% or more identical or similar to a nucleotide sequence as defined in SEQ ID NO:1, 2, or 3, or a portion thereof. The length of the sequence comparison for nucleic acids is at least 50 consecutive nucleotides, or at least 100 consecutive nucleotides, or at least 200 consecutive nucleotides. In yet another embodiment, an isolated nucleic acid of the invention is complementary to a polynucleotide as defined in SEQ ID NO:1, 2, or 3, or to a polynucleotide having 70% sequence identity to a polynucleotide as defined in SEQ ID NO:1, 2, or 3, or to a polynucleotide hybridizing under stringent conditions to a polynucleotide as defined in SEQ ID NO:1, 2, or 3.

In another embodiment, homologs of the specific transcription regulatory elements may include, but are not limited to, polynucleotides comprising deletions of nucleotide fragments, single or multiple point mutations, alterations at a particular restriction enzyme site, addition or rearrangement of functional elements, or other means of molecular modification. This modification may or may not enhance, or otherwise alter the transcription regulatory activity of said nucleic acid. For example, one of skill in the art may identify the functional elements within the sequences and delete any non-essential elements. Functional elements may be modified, combined or rearranged to increase the utility or expression of the polynucleotides of the invention for any particular application. Functionally equivalent fragments of a transcription regulatory nucleotide sequence of the invention can also be obtained by removing or deleting non-essential sequences without deleting the essential one. Narrowing the transcription regulating nucleotide sequence to its essential and transcription mediating elements can be realized in vitro by trial-and-error deletion mutations, or in silico using promoter element search routines. Regions essential for promoter activities often demonstrate clusters of certain and known promoter elements. Such analysis can be performed using available computer algorithms, such as, PLACE (“Plant Cis-acting Regulatory DNA Elements”; Higo 1999), the BIOBASE database “Transfac” (Biologische Datenbanken GmbH, Braunschweig; Wingender 2001) or the database PlantCARE (Lescot 2002). Equivalent fragments of transcription regulating nucleotide sequences can also be obtained by deleting the region encoding the 5′-untranslated region of the mRNA, thus only providing the (untranscribed) promoter region. The 5′-untranslated region can be easily determined by methods known in the art (such as 5′-RACE analysis). Accordingly, some of the transcription regulatory nucleotide sequences of the invention are equivalent fragments of other sequences.

The present invention contemplates that in addition to the specific polynucleotide as defined in SEQ ID NO:1, 2, or 3, its specific elements, homologs of polynucleotide as defined in SEQ ID NO:1, 2, or 3 can be employed. As used herein, the term “analogs” refers to genes that have the same or similar function, but that have evolved separately in unrelated organisms. The term “homologs” as used herein refers to a gene related to a second gene by descent from a common ancestral DNA sequence. The term “homologs” may apply to the relationship between genes separated by the event of speciation (e.g., orthologs) or to the relationship between genes separated by the event of genetic duplication (e.g., paralogs). The term “orthologs” refers to genes from different species, but that have evolved from a common ancestral gene by speciation. Orthologs retain the same function in the course of evolution. Orthologs encode proteins having the same or similar functions. As used herein, the term “paralogs” refers to genes that are related by duplication within a genome. Paralogs usually have different functions or new functions, but these functions may be related.

Another subset of homologs is allelic variant. As used herein, the term “allelic variant” refers to a nucleotide containing polymorphisms that lead to changes in the amino acid sequences of a protein encoded by the nucleotide and that exist within a natural population (e.g., a plant species or variety). Such natural allelic variations can typically result in 1-5% variance in a polynucleotide encoding a protein. Allelic variants can be identified by sequencing the nucleic acid of interest in a number of different plants, which can be readily carried out by using, for example, hybridization probes to identify the same gene genetic locus in those plants. Any and all such nucleic acid variations and resulting amino acid polymorphisms or variations in a nucleic acid of the invention that are the result of natural allelic variation and that do not alter the functional activity of the encoded protein, are intended to be within the scope of the invention.

The term “sequence alignment” used herein refers to a method of arranging the primary sequences of DNA, RNA, or protein to identify regions of similarity that may be a consequence of functional, structural, or evolutionary relationships between the sequences. Computational approaches to sequence alignment generally fall into two categories: global alignments and local alignments. A global alignment is a form of global optimization that forces the alignment to span the entire length of all query sequences. A local alignment identifies regions of similarity within long sequences that are often widely divergent overall.

The term “conserved region” or “conserved domain” as used herein refers to a region in heterologous polynucleotide or polypeptide sequences where there is a relatively high degree of sequence identity between the distinct sequences. The “conserved region” can be identified, for example, from the multiple sequence alignment using the Clustal W algorithm.

As used herein, the term “sequence identity” or “identity” in the context of two nucleic acid or polypeptide sequences makes reference to the residues in the two sequences that are the same when aligned for maximum correspondence over a specified comparison window, for example, either the entire sequence as in a global alignment or the region of similarity in a local alignment. When percentage of sequence identity is used in reference to proteins it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skilled in the art. Typically this involves scoring a conservative substitution as a partial rather than a full mismatch, thereby increasing the percentage of sequence similarity.

As used herein, “percentage of sequence identity” or “sequence identity percentage” means the value determined by comparing two optimally aligned sequences over a comparison window, either globally or locally, wherein the portion of the sequence in the comparison window may comprise gaps for optimal alignment of the two sequences. In principle, the percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. “Percentage of sequence similarity” for protein sequences can be calculated using the same principle, wherein the conservative substitution is calculated as a partial rather than a complete mismatch. Thus, for example, where an identical amino acid is given a score of 1 and a non-conservative substitution is given a score of zero, a conservative substitution is given a score between zero and 1. The scoring of conservative substitutions can be obtained from amino acid matrices known in the art, for example, Blosum or PAM matrices.

Methods of alignment of sequences for comparison are well known in the art. The determination of percent identity or percent similarity (for proteins) between two sequences can be accomplished using a mathematical algorithm. Preferred, non-limiting examples of such mathematical algorithms are, the algorithm of Myers and Miller (Bioinformatics, 4(1):11-17, 1988), the Needleman-Wunsch global alignment (J Mol Biol. 48(3):443-53, 1970), the Smith-Waterman local alignment (Journal of Molecular Biology, 147:195-197, 1981), the search-for-similarity-method of Pearson and Lipman (PNAS, 85(8): 2444-2448, 1988), the algorithm of Karlin and Altschul (J. Mol. Biol., 215(3):403-410, 1990; PNAS, 90:5873-5877, 1993).

Computer implementations of these mathematical algorithms can be utilized for comparison of sequences to determine sequence identity or to identify homologs. Such implementations include, but are not limited to, the programs described below.

The BLAST (Basic Local Alignment Search Tool) programs have been designed for speed to find high scoring local alignments. BLAST uses a heuristic algorithm that seeks local as opposed to global alignments and is therefore able to detect relationships among sequences which share only isolated regions of similarity (Altschul et al, 1990 and 1993). The BLAST programs contain a few individual programs: BLASTN compares a nucleotide query sequence against a nucleotide sequence database, BLASTP compares a protein query sequence against a protein sequence database, BLASTX compares the six-frame conceptual translation products of a nucleotide query sequence (both strands) against a protein sequence database, TBLASTN takes a protein sequence and compares it against a nucleotide database which has been translated in all six reading frames, TBLASTX converts a nucleotide query sequence into protein sequences in all 6 reading frames and then compares this to a nucleotide database which has been translated on all six reading frames. Gapped BLAST can be utilized to obtain gapped alignments for comparison purposes (Nucleic Acids Research, 25(17):3389-3402, 1997). Alternatively, PSI-BLAST can be used to perform an iterated search that detects distant relationships between molecules. Software for performing BLAST analyses is publicly available through the National Center for Biotechnology Information website.

In addition to calculating percent sequence identity or percent sequence similarity, the BLAST algorithm also performs a statistical analysis of the similarity between two sequences (Altschul et al., 1993). One measure of similarity provided by the BLAST algorithm is the smallest sum probability (P(N)), also called the P-value, which provides an indication of the probability by which a match between two nucleotides or amino acid sequences would occur by chance. Another measure is the Expect value (E-value) which represents the number of times this match or a better one would be expected to occur purely by chance in a search of the entire database. Thus, the lower the E-value, the greater the similarity is between the input sequence and the match sequence.

FASTA is a DNA and protein sequence alignment software package developed based on the Pearson and Lipman algorithm. The FASTA package contains programs for protein:protein, DNA:DNA, protein:translated DNA (with frameshifts), and ordered or unordered peptide searches. The FASTA package is available through the University of Virginia website.

Multiple alignments (e.g., of more than two DNA or protein sequences) can be performed using the ClustalW algorithm (Thompson et. al. Nucleic Acids Res. 22:4673-4680, 1994) as implemented in, for example, Vector NTI package (Invitrogen, 1600 Faraday Ave., Carlsbad, Calif. 92008).

It is well known in the art that one or more amino acids in a native sequence can be substituted with another amino acid(s), the charge and polarity of which are similar to that of the native amino acid, i.e., a conservative amino acid substitution. Conserved substitutions for an amino acid within the native polypeptide sequence can be selected from other members of the class to which the naturally occurring amino acid belongs. Amino acids can be divided into the following four groups: (1) acidic amino acids, (2) basic amino acids, (3) neutral polar amino acids, and (4) neutral nonpolar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine.

Transcription regulatory elements of the invention may be isolated from plants other than maize or rice using the information provided herein and techniques known to those of skilled in the art of biotechnology. For example, a polynucleotide encoding a starch synthase can be isolated from plant tissue cDNA libraries, wherein the plant can be selected from the group consisting of wheat, barley, sorghum, rye, triticale, maize, rice, sugarcane, pea, alfalfa, soybean, carrot, celery, tomato, potato, cotton, tobacco, pepper, oilseed rape, beet, cabbage, cauliflower, broccoli, lettuce and Arabidopsis thaliana. Synthetic oligonucleotide primers for polymerase chain reaction amplification can be designed based upon the sequence of aforementioned starch synthase encoding polynucleotide. The transcription regulatory element upstream of the polynucleotide, or downstream of the polynucleotide, or in the intron regions can be isolated using the corresponding plant genomic DNA as a template in PCR. Alternatively, a polynucleotide from a plant that hybridizes under stringent conditions to a polynucleotide as defined in SEQ ID NO:1, 2, or 3, or a polynucleotide having at least 70-80%, 80-85%, 85-90% or 90-95%, or at least about 95%, 96%, 97%, 98%, 99% or more identical or similar to a polynucleotide as defined in SEQ ID NO:1, 2, or 3 can be isolated from plant tissue genomic DNA libraries. The transcription regulatory elements derived from said polynucleotide can be isolated in any number of standard ways such as by PCR as described above. The transcription regulatory elements so isolated can be cloned into appropriate vectors and characterized by DNA sequence analysis.

In another embodiment, the transcription regulating activity of a homolog of the transcription regulatory nucleotides is substantially the same (or equivalent) to the transcription regulatory nucleotide specifically disclosed herein, i.e. that expression is regulated in the endosperm-preferred fashion as described above. In addition to this, the transcription regulatory activity of a homolog may vary from the activity of its parent polynucleotide, especially with respect to expression level. The expression level may be higher or lower than the expression level of the parent polynucleotides. Both derivations may be advantageous depending on the nucleic acid of interest to be expressed. Preferred are such functional equivalent polynucleotide, which—in comparison with its parent nucleotide—does not deviate from the expression level of said parent polynucleotide by more than 50%, or 25%, or 10%. Also preferred are equivalent polynucleotides which demonstrate an increased expression in comparison to their parent polynucleotide, an increase by at least 50%, or at least 100%, or at least 500%. The expression level can be judged by either mRNA expression or protein expression. Protein expression profile can be demonstrated using reporter genes operably linked to said transcription regulatory polynucleotide. Preferred reporter genes (Schenborn 1999) in this context are green fluorescence protein (GFP) (Chui 1996; Leffel 1997), chloramphenicol transferase, luciferase (Millar 1992), β-glucuronidase or β-galactosidase (Jefferson 1987). The methods to assay transcriptional regulation are well known in the art, and include Northern blots and RT-PCR.

Another embodiment of the invention relates to an isolated plant transcription terminator element comprising a polynucleotide selected from the group consisting of: a) a polynucleotide having a sequence as defined in SEQ ID NO:4 or SEQ ID NO:5; b) a polynucleotide having 70% sequence identity to a polynucleotide having a sequence as defined in SEQ ID NO:4 or SEQ ID NO:5; c) a polynucleotide having a fragment of at least 50 consecutive nucleotides, or at least 100 consecutive nucleotides, or at least 200 consecutive nucleotides of a polynucleotide having a sequence as defined in SEQ ID NO:4 or SEQ ID NO:5; d) a polynucleotide hybridizing under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:4 or SEQ ID NO:5; and e) a polynucleotide complementary to any of the polynucleotides of a) through d).

In another embodiment, an isolated plant transcription terminator element of the invention comprises a nucleotide sequence which is at least about 50-60%, or at least about 60-70%, or at least about 70-80%, 80-85%, 85-90% or 90-95%, or at least about 95%, 96%, 97%, 98%, 99% or more identical or similar to a nucleotide sequence as defined in SEQ ID NO:4 or 5, or a portion thereof. The length of the sequence comparison for nucleic acids is at least 50 consecutive nucleotides, or at least 100 consecutive nucleotides, or at least 200 consecutive nucleotides. In yet another embodiment, an isolated nucleic acid of the invention is complementary to a polynucleotide as defined in SEQ ID NO:4 or 5, or to a polynucleotide having 70% sequence identity to a polynucleotide as defined in SEQ ID NO:4 or 5, or to a polynucleotide hybridizing under stringent conditions to a polynucleotide as defined in SEQ ID NO:4 or 5.

In another embodiment, an isolated nucleic acid hybridizes under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:4 or 5, or a portion thereof. In another embodiment, the conditions are such that sequences at least about 60%, or at least about 65%, or at least about 70%, or at least about 75% or more similar or identical to each other typically remain hybridized to each other. Such stringent conditions are known to those skilled in the art and as described above. A preferred, non-limiting example of stringent conditions are hybridization in 6× sodium chloride/sodium citrate (SSC) at about 45° C., followed by one or more washes in 0.2×SSC, 0.1% SDS at 50-65° C.

Another embodiment of the invention relates to a plant transformed with an isolated plant transcription regulatory element comprising a polynucleotide, wherein the polynucleotide is selected from the group consisting of: a) a polynucleotide having a sequence as defined in SEQ ID NO:1, 2, or 3; b) a polynucleotide having 70% sequence identity to a polynucleotide having a sequence as defined in SEQ ID NO:1, 2, or 3; c) a polynucleotide having a fragment of at least 50 consecutive nucleotides, or at least 100 consecutive nucleotides, or at least 200 consecutive nucleotides of a polynucleotide having a sequence as defined in SEQ ID NO:1, 2, or 3; d) a polynucleotide hybridizing under stringent conditions to a polynucleotide comprising at least 50 consecutive nucleotides, or at least 100 consecutive nucleotides, or at least 200 consecutive nucleotides of a polynucleotide having the sequence as defined in SEQ ID NO:1, 2, or 3; and e) a polynucleotide complementary to any of the polynucleotides of a) through d).

Yet another embodiment of the invention relates to a plant transformed with an isolated plant transcription regulatory element comprising a polynucleotide operably linked to one or more heterologous nucleic acids, wherein said plant transcription regulatory element comprising a polynucleotide is selected from the group consisting of: a) a polynucleotide having a sequence as defined in SEQ ID NO:1, 2, or 3; b) a polynucleotide having 70% sequence identity to a polynucleotide having a sequence as defined in SEQ ID NO:1, 2, or 3; c) a polynucleotide having a fragment of at least 50 consecutive nucleotides, or at least 100 consecutive nucleotides, or at least 200 consecutive nucleotides of a polynucleotide having a sequence as defined in SEQ ID NO:1, 2, or 3; d) a polynucleotide hybridizing under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:1, 2, or 3; and e) a polynucleotide complementary to any of the polynucleotides of a) through d).

In one embodiment, the transformed plant may be a plant selected from the group consisting of monocotyledonous and dicotyledonous plants. The plant can be from a genus selected from the group consisting of maize, wheat, rice, barley, oat, rye, sorghum, banana, and ryegrass. The plant can be from a genus selected from the group consisting of pea, alfalfa, soybean, carrot, celery, tomato, potato, cotton, tobacco, pepper, oilseed rape, beet, cabbage, cauliflower, broccoli, lettuce and Arabidopsis thaliana. In another embodiment, the transformed plant expresses an agronomically relevant or phenotypic trait. Such traits include, but not limited to, oil quantity and quality, protein quality and quantity, amino acid composition, starch quality and quantity, increased feed content and value, increased food content and value, increased yield, increased stress tolerance or resistance, such as resistance or tolerance to drought, heat, chilling, freezing, excessive moisture, salt, oxidative stress, and nitrogen stress, herbicide resistance or tolerance, insect resistance or tolerance, disease resistance or tolerance, physical appearance, male sterility, female sterility, and the like.

The present invention also provides a monocotyledonous or dicotyledonous transformed plant, seed and parts from such a plant, and progeny plants from such a plant, including hybrids and inbreds. Another embodiment of the invention provides a seed produced by a transgenic plant transformed with an expression vector comprising a plant transcription regulatory element of the present invention. The seed is true breeding for the plant transcription regulatory element, wherein the transcription regulatory element regulates endosperm-specific expression of an exogenous or endogenous gene. The invention also provides a method of plant breeding, e.g., to prepare a crossed fertile transgenic plant. The method comprises crossing a fertile transgenic plant comprising a particular expression vector of the invention with itself or with a second plant, e.g., one lacking the particular expression vector, to prepare the seed of a crossed fertile transgenic plant comprising the particular expression vector. The seed is then planted to obtain a crossed fertile transgenic plant. The plant may be a monocot or dicot. The crossed fertile transgenic plant may have the present expression vector inherited through a female parent or through a male parent. The second plant may be an inbred plant. The crossed fertile transgenic may be a hybrid. Also included within the present invention are seeds of any of these crossed fertile transgenic plants.

One of the most economically relevant traits is increased nutritional value in a plant. Accordingly, another embodiment of the invention relates to a method for conferring increased nutritional value, increased nutritional quality, increased or modified starch content, increased or modified oil content of a seed or a sprout of a plant, said method comprises the steps of:
1) introducing into a plant cell or a plant an expression vector, wherein the expression vector comprises:
   A) a plant transcription regulatory element comprising a polynucleotide selected from the group consisting of:
      a) a polynucleotide having a sequence as defined in SEQ ID NO:1, 2, or 3;
      b) a polynucleotide having 70% sequence identity to a polynucleotide having a sequence as defined in SEQ ID NO:1, 2, or 3;
      c) a polynucleotide having a fragment of at least 50 consecutive nucleotides, or at least 100 consecutive nucleotides, or at least 200 consecutive nucleotides of a polynucleotide having a sequence as defined in SEQ ID NO:1, 2, or 3;
      d) a polynucleotide hybridizing under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:1, 2, or 3; and
      e) a polynucleotide complementary to any of the polynucleotides of a) through d); and operably linked thereto one or more heterologous nucleic acids, wherein the operably linked nucleic acid encodes a polypeptide that is capable to confer to a plant increased nutritional value and/or increased or modified starch content to the plant;
   B) selecting transgenic plants, wherein the plants have increased nutritional value, increased nutritional quality, increased or modified starch content, increased or modified oil content of a seed or a sprout of the plants, as compared to the wild type or null segregant plants.

The nutritional value may comprise protein quality and quantity, oil quality and quantity, amino acid composition, starch quality and quantity, feed content and value, food content and value, and content of at least one compound selected from the group consisting of vitamins, carotinoids, antioxidants, unsaturated fatty acids and poly-unsaturated fatty acids. The nutritional value and the corresponding nucleic acid to be expressed are defined herein below. Preferred plant transcription regulatory elements are described above. The plant to which the methods of this invention are applied to may be selected from the group consisting of maize wheat, rice barley, oat rye, sorghum, banana, ryegrass, pea, alfalfa, soybean, carrot, celery, tomato, potato, cotton, tobacco, pepper, oilseed rape, beet, cabbage, cauliflower, broccoli, lettuce and Arabidopsis thaliana.

An increased nutritional value may, for example, result in one or more of the following properties: modified fatty acid composition in a plant, altered amino acid content of a plant, increased concentration of a plant metabolite. A wide range of novel transgenic plants produced in this manner may be envisioned depending on the particular end use of the grain.

For example, the largest use of maize grain is for feed or food. Introduction of genes that alter the composition of the grain may greatly enhance the feed or food value. The primary components of maize grain are starch, protein, and oil. Each of these primary components of maize grain may be improved by altering its level or composition. Several examples may be mentioned for illustrative purposes but in no way provide an exhaustive list of possibilities.

The protein of many cereal grains is suboptimal for feed and food purposes, especially when fed to pigs, poultry, and humans. The protein is deficient in several amino acids that are essential in the diet of these species, requiring the addition of supplements to the grain. These essential amino acids may include lysine, methionine, tryptophan, threonine, valine, arginine, and histidine. Some amino acids become limiting only after the grain is supplemented with other inputs for feed formulations. For example, when the grain is supplemented with soybean meal to meet lysine requirements, methionine becomes limiting. The levels of these essential amino acids in seeds and grain may be elevated by mechanisms which include, but are not limited to, the introduction of genes to increase the biosynthesis of the amino acids, decrease the degradation of the amino acids, increase the storage of the amino acids in proteins, or increase transport of the amino acids to the seeds or grain.

One mechanism for increasing the biosynthesis of the amino acids is to introduce genes that deregulate the amino acid biosynthetic pathways such that the plant can no longer adequately control the levels of the amino acids that are produced. This may be done by deregulating or bypassing steps in the amino acid biosynthetic pathway that are normally regulated by levels of the amino acid end product of the pathway. Examples include the introduction of genes that encode deregulated versions of the enzymes aspartokinase or dihydrodipicolinic acid (DHDP)-synthase for increasing lysine and threonine production, and anthranilate synthase for increasing tryptophan production. Reduction of the catabolism of the amino acids may be accomplished by introduction of DNA sequences that reduce or eliminate the expression of genes encoding enzymes that catalyse steps in the catabolic pathways such as the enzyme lysine-ketoglutarate reductase.

The protein composition of the grain may be altered to improve the balance of amino acids in a variety of ways, including elevating expression of native proteins, decreasing expression of those with poor composition, changing the composition of native proteins, or introducing genes encoding entirely new proteins that possess superior composition. DNA may be introduced that decreases the expression of members of the zein family of storage proteins in maize. This DNA may encode ribozymes or antisense sequences directed to impair expression of zein proteins or expression of regulators of zein expression such as the opaque-2 gene product. The protein composition of the grain may be modified through cosuppression, i.e., inhibition of expression of an endogenous gene through the expression of an identical structural gene or gene fragment introduced through transformation (Goring 1991). Additionally, the introduced DNA may encode enzymes, which degrade zeins. The decreases in zein expression that are achieved may be accompanied by increases in proteins with more desirable amino acid composition or increases in other major seed constituents such as starch. Alternatively, a chimeric gene may be introduced that comprises a coding sequence for a native protein of adequate amino acid composition such as for one of the globulin proteins or 10 kD zein of maize and a promoter or other regulatory sequence designed to elevate expression of said protein. The coding sequence of said gene may include additional or replacement codons for essential amino acids. Further, a coding sequence obtained from another species, or, a partially or completely synthetic sequence encoding a completely unique peptide sequence designed to enhance the amino acid composition of the seed may be employed.

The introduction of genes that alter the oil content of the grain may be of value. Increases in oil content may result in increases in metabolizable energy content and desired density of the seeds for uses in feed and food. The introduced genes may encode enzymes that remove or reduce rate-limitations or regulated steps in fatty acid or lipid biosynthesis. Such genes may include, but are not limited to, those that encode acetyl-CoA carboxylase, ACP-acyltransferase, beta-ketoacyl-ACP synthase, plus other well-known proteins involved in fatty acid biosynthetic activities. Other possibilities are genes that encode proteins that do not possess enzymatic activity such as acyl carrier protein. Additional examples include 2-acetyltransferase, oleosin pyruvate dehydrogenase complex, acetyl CoA synthetase, ATP citrate lyase, ADP-glucose pyrophosphorylase and genes of the carnitine-CoA-acetyl-CoA shuttles. Genes may be introduced that alter the balance of fatty acids present in the oil providing a healthier or nutritious feedstuff. The introduced DNA may also encode sequences that block expression of enzymes involved in fatty acid biosynthesis, altering the proportions of fatty acids present in the grain such as described below.

In addition to altering the major constituents of the grain, genes may be introduced that affect a variety of other nutrients, processing steps, or other quality aspects of the grain as used for feed or food. For example, pigmentation of the grain may be increased or decreased. Enhancement and stability of yellow pigmentation is desirable in some animal feeds and may be achieved by introduction of genes that result in enhanced production of xanthophylls and carotenes by eliminating rate-limiting steps in their production. Such genes may encode altered forms of the enzymes phytoene synthase, phytoene desaturase, or lycopene synthase. Alternatively, unpigmented white corn is desirable for production of many food products and may be produced by the introduction of DNA, which blocks or eliminates steps in pigment production pathways.

Feed or food comprising some cereal grains possesses insufficient quantities of vitamins and must be supplemented to provide adequate nutritional value. Introduction of genes that enhance vitamin biosynthesis in seeds may be envisioned including, for example, vitamins A, E, B₁₂, choline, and the like. For example, maize grain also does not possess sufficient mineral content for optimal nutritional value. Genes that affect the accumulation or availability of compounds containing phosphorus, sulfur, calcium, manganese, zinc, and iron among others would be valuable. An example may be the introduction of a gene that reduces phytic acid production or encodes the enzyme phytase, which enhances phytic acid breakdown. These genes would increase levels of available phosphate in the diet, reducing the need for supplementation with mineral phosphate.

In addition to direct improvements in feed or food value, genes may also be introduced which improve the processing of grain and improve the value of the products resulting from the processing. The primary method of processing certain grains such as maize is via wetmilling. Maize may be improved through the expression of novel genes that increase the efficiency and reduce the cost of processing such as by decreasing steeping time.

Improving the value of wetmilling products may include altering the quantity or quality of starch, oil, corn gluten meal, or the components of corn gluten feed. Elevation of starch may be achieved through the identification and elimination of rate limiting steps in starch biosynthesis or by decreasing levels of the other components of the grain resulting in proportional increases in starch. An example of the former may be the introduction of genes encoding ADP-glucose pyrophosphorylase enzymes with altered regulatory activity or which are expressed at higher level. Examples of the latter may include selective inhibitors of, for example, protein or oil biosynthesis expressed during later stages of kernel development.

Oil is another product of wetmilling of corn and other grains, the value of which may be improved by introduction and expression of genes. The quantity of oil that can be extracted by wetmilling may be elevated by approaches as described for feed and food above. Oil properties may also be altered to improve its performance in the production and use of cooking oil, shortenings, lubricants or other oil-derived products or improvement of its health attributes when used in the food-related applications. Novel fatty acids may also be synthesized which upon extraction can serve as starting materials for chemical syntheses. The changes in oil properties may be achieved by altering the type, level, or lipid arrangement of the fatty acids present in the oil. This in turn may be accomplished by the addition of genes that encode enzymes that catalyze the synthesis of novel fatty acids or by increasing levels of native fatty acids while possibly reducing levels of precursors. Alternatively nucleic acids may be introduced which slow or block steps in fatty acid biosynthesis resulting in the increase in precursor fatty acid intermediates. Genes that might be added include desaturases, epoxidases, hydratases, dehydratases, and other enzymes that catalyze reactions involving fatty acid intermediates. Representative examples of catalytic steps that might be blocked include the desaturations from stearic to oleic acid and oleic to linolenic acid resulting in the respective accumulations of stearic and oleic acids.

Improvements in the other major cereal wetmilling products, gluten meal and gluten feed, may also be achieved by the introduction of genes to obtain novel plants. Representative possibilities include, but are not limited to, those described above for improvement of food and feed value.

In addition, it may further be considered that the plant be used for the production or manufacturing of useful biological compounds that were either not produced at all, or not produced at the same level, in the plant previously. The novel plants producing these compounds are made possible by the introduction and expression of genes by transformation methods. The possibilities include, but are not limited to, any biological compound which is presently produced by any organism, such as proteins, nucleic acids, primary and intermediate metabolites, carbohydrate polymers, etc. The compounds may be produced by the plant, extracted upon harvest and/or processing, and used for any presently recognized useful purpose such as pharmaceuticals, fragrances, industrial enzymes, to name a few.

Useful nucleic acids that can be combined with the transcription regulatory elements of the present invention and provide improved end-product traits include, without limitation, those encoding seed storage proteins, fatty acid pathway enzymes, tocopherol biosynthetic enzymes, amino acid biosynthetic enzymes, and starch branching enzymes. A discussion of exemplary genes useful for the modification of plant phenotypes may be found in, for example, U.S. Pat. Nos. 6,194,636; 6,207,879; 6,232,526; 6,426,446; 6,429,357; 6,433,252; 6,437,217; 6,515,201; and 6,583,338 and also in WO 02/057471, each of which is specifically incorporated herein by reference in its entirety. Such traits include but are not limited to:
Expression of metabolic enzymes for use in the food-and-feed sector, for example, phytases and cellulases. Especially preferred are nucleic acids such as the artificial cDNA which encodes a microbial phytase (GenBank Acc. No. A19451) or functional equivalents thereof.
Expression of genes which bring about an accumulation of fine chemicals such as tocopherols, tocotrienols or carotenoids. An example which may be mentioned is phytoene desaturase. Preferred are nucleic acids which encode the Narcissus pseudonarcissus photoene desaturase (GenBank Acc. No. X78815) or functional equivalents thereof. Preferred tocopherol biosynthetic enzymes include tyrA, slr1736, ATPT2, dxs, dxr, GGPPS, HPPD, GMT, MT1, tMT2, AANT1, sir 1737, and an antisense construct for homogentisic acid dioxygenase (Kridl et al. (1991); Keegstra (1989); Nawrath et al. (1994); Xia et al. (1992); Lois et al. (1998); Takahashi et al. (1998); Norris et al. (1998); Bartley and Scolnik (1994); Smith et al. (1997); WO 00/32757; WO 00/10380; Saint Guily et al. (1992); Sato et al. (2000), all of which are incorporated herein by reference.
Starch production (U.S. Pat. Nos. 5,750,876 and 6,476,295), high protein production (U.S. Pat. No. 6,380,466), fruit ripening (U.S. Pat. No. 5,512,466), enhanced animal and human nutrition (U.S. Pat. Nos. 5,985,605 and 6,171,640), biopolymers (U.S. Pat. No. 5,958,745 and U.S. Patent Publication No. 2003/0028917), environmental stress resistance (U.S. Pat. No. 6,072,103), pharmaceutical peptides (U.S. Pat. No. 6,080,560), improved processing traits (U.S. Pat. No. 6,476,295), improved digestibility (U.S. Pat. No. 6,531,648), low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), and biofuel production (U.S. Pat. No. 5,998,700), the genetic elements and transgenes described in the patents listed above are herein incorporated by reference. Preferred starch branching enzymes (for modification of starch properties) include those set forth in U.S. Pat. Nos. 6,232,122 and 6,147,279; and WO 97/22703, all of which are incorporated herein by reference.
Modified oils production (U.S. Pat. No. 6,444,876), high oil production (U.S. Pat. Nos. 5,608,149 and 6,476,295), or modified fatty acid content (U.S. Pat. No. 6,537,750). Preferred fatty acid pathway enzymes include thioesterases (U.S. Pat. Nos. 5,512,482; 5,530,186; 5,945,585; 5,639,790; 5,807,893; 5,955,650; 5,955,329; 5,759,829; 5,147,792; 5,304,481; 5,298,421; 5,344,771; and 5,760,206), diacylglycerol acyltransferases (U.S. Patent Publications 20030115632 and 20030028923), and desaturases (U.S. Pat. Nos. 5,689,050; 5,663,068; 5,614,393; 5,856,157; 6,117,677; 6,043,411; 6,194,167; 5,705,391; 5,663,068; 5,552,306; 6,075,183; 6,051,754; 5,689,050; 5,789,220; 5,057,419; 5,654,402; 5,659,645; 6,100,091; 5,760,206; 6,172,106; 5,952,544; 5,866,789; 5,443,974; and 5,093,249) all of which are incorporated herein by reference.
Preferred amino acid biosynthetic enzymes include anthranilate synthase (U.S. Pat. No. 5,965,727 and WO 97/26366, WO 99/11800, WO 99/49058), tryptophan decarboxylase (WO 99/06581), threonine decarboxylase (U.S. Pat. Nos. 5,534,421 and 5,942,660; WO 95/19442), threonine deaminase (WO 99/02656 and WO 98/55601), dihydrodipicolinic acid synthase (U.S. Pat. No. 5,258,300), and aspartate kinase (U.S. Pat. Nos. 5,367,110; 5,858,749; and 6,040,160) all of which are incorporated herein by reference.
Production of nutraceuticals such as, for example, polyunsaturated fatty acids (for example arachidonic acid, eicosapentaenoic acid or docosahexaenoic acid) by expression of fatty acid elongases and/or desaturases, or production of proteins with improved nutritional value such as, for example, with a high content of essential amino acids (for example the high-methionine 2S albumin gene of the brazil nut). Preferred are nucleic acids which encode the Bertholletia excelsa high-methionine 2S albumin (GenBank Acc. No. AB044391), the Physcomitrella patens Δ6-acyl-lipid desaturase (GenBank Acc. No. AJ222980; Girke et al. 1998), the Mortierella alpina Δ6-desaturase (Sakuradani et al. 1999), the Caenorhabditis elegans Δ5-desaturase (Michaelson et al. 1998), the Caenorhabditis elegans Δ5-fatty acid desaturase (des-5) (GenBank Acc. No. AF078796), the Mortierella alpina Δ5-desaturase (Michaelson et al. JBC 273:19055-19059), the Caenorhabditis elegans Δ6-elongase (Beaudoin et al. 2000), the Physcomitrella patens Δ6-elongase (Zank et al. 2000), or functional equivalents of these.
Production of high-quality proteins and enzymes for industrial purposes (for example enzymes, such as lipases) or as pharmaceuticals (such as, for example, antibodies, blood clotting factors, interferons, lymphokins, colony stimulation factor, plasminogen activators, hormones or vaccines, as described by Hood E E and Jilka J M 1999). For example, it has been possible to produce recombinant avidin from chicken albumen and bacterial β-glucuronidase (GUS) on a large scale in transgenic maize plants (Hood et al. 1999).

Obtaining an increased storability in cells which normally comprise fewer storage proteins or storage lipids, with the purpose of increasing the yield of these substances, for example by expression of acetyl-CoA carboxylase. Preferred nucleic acids are those which encode the Medicago sativa acetyl-CoA carboxylase (ACCase) (GenBank Acc. No. L25042), or functional equivalents thereof. Alternatively, in some scenarios an increased storage protein content might be advantageous for high-protein product production. Preferred seed storage proteins include zeins (U.S. Pat. Nos. 4,886,878; 4,885,357; 5,215,912; 5,589,616; 5,508,468; 5,939,599; 5,633,436; and 5,990,384; WO 90/01869, WO 91/13993, WO 92/14822, WO 93/08682, WO 94/20628, WO 97/28247, WO 98/26064, and WO 99/40209), 7S proteins (U.S. Pat. Nos. 5,003,045 and 5,576,203), brazil nut protein (U.S. Pat. No. 5,850,024), phenylalanine free proteins (WO 96/17064), albumin (WO 97/35023), beta-conglycinin (WO 00/19839), 11S (U.S. Pat. No. 6,107,051), alpha-hordothionin (U.S. Pat. Nos. 5,885,802 and 5,885,801), arcelin seed storage proteins (U.S. Pat. No. 5,270,200), lectins (U.S. Pat. No. 6,110,891), and glutenin (U.S. Pat. Nos. 5,990,389 and 5,914,450) all of which are incorporated herein by reference.

Further examples of advantageous genes are mentioned, for example, in Dunwell J M, Transgenic approaches to crop improvement, J Exp Bot. 2000; 51 Spec No; pages 487-96. A discussion of exemplary DNAs useful for the modification of plant phenotypes may be found in, for example, U.S. Pat. No. 6,194,636.

Another aspect of the invention provides a DNA construct in which the promoter with starchy-endosperm and/or germinating embryo-specific or -preferred expression drives a gene suppression DNA element, e.g. to suppress an amino acid catabolizing enzyme.

Seed-specific or endosperm-preferred promoters of this invention may be useful in minimizing yield drag and other potential adverse physiological effects on maize growth and development that might be encountered by high-level, non-inducible, constitutive expression of a transgenic protein or other molecules in a plant. When each transgene is fused to a promoter of the invention, the risk of DNA sequence homology dependent transgene inactivation (co-suppression) can be minimized.

It may be useful to target DNA itself within a cell. For example, it may be useful to target introduced DNA to the nucleus as this may increase the frequency of transformation. Within the nucleus itself it would be useful to target a gene in order to achieve site-specific integration. For example, it would be useful to have a gene introduced through transformation to replace an existing gene in the cell, or to introduce a nucleic acid with regulatory function to a specific location on the genome to regulate the expression of a endogenous gene of interest. Other elements include those that can be regulated by endogenous or exogenous agents, e.g., by zinc finger proteins, including naturally occurring zinc finger proteins or chimeric zinc finger proteins (see, e.g., U.S. Pat. No. 5,789,538, WO 99/48909; WO 99/45132; WO 98/53060; WO 98/53057; WO 98/53058; WO 00/23464; WO 95/19431; and WO 98/54311) or myb-like transcription factors. For example, a zinc finger protein may include a DNA recognition domain that binds to a specific DNA sequence (the zinc finger) and a functional domain that activates (e.g., GAL 4 sequences) or represses a target nucleic acid.

General categories of genes of interest for the purposes of the present invention include, for example, those genes involved in information, such as Zinc fingers, those involved in communication, such as kinases, and those involved in housekeeping, such as heat shock proteins. More specific categories of transgenes include genes encoding important traits for agronomic quality, insect resistance, disease resistance, herbicide resistance, and grain characteristics. Still other categories of transgenes include genes for inducing expression of exogenous products such as enzymes, cofactors, and hormones from plants and other eukaryotes as well as from prokaryotic organisms.

The transcription regulatory elements of the invention may modulate genes encoding proteins which act as cell cycle regulators, or which control carbohydrate metabolism or phytohormone levels, as has been shown in tobacco and canola with other tissue-preferred promoters. (Ma, Q. H. et al., 1998; Roeckel, P. et al., 1997) For example, genes encoding isopentenyl transferase or IAA-M may be useful in modulating development of the female florets. Other important genes encode growth factors and transcription factors. Expression of selected endogenous or heterologous nucleotides under the direction of the promoter may result in continued or improved development of the female florets under adverse conditions.

Another economically relevant trait is yield. Yield is heavily affected by damage to the embryo, endosperm or young seedling. Accordingly, any kind of trait which protects the young seedling, embryo or endosperm or enhances its performance is advantageous with respect to yield. Thus, a trait resulting in stress resistance can also result in increased yield. Another embodiment of the invention relates to a method for conferring increased yield and/or increased stress tolerance to a plant, said method comprises the steps of:
A) introducing into a plant cell or a plant an expression vector, wherein the expression vector comprises:
   1) a plant transcription regulatory element comprising a polynucleotide selected from the group consisting of:
      a) a polynucleotide having a sequence as defined in SEQ ID NO:1, 2 or 3;
      b) a polynucleotide having 70% sequence identity to a polynucleotide having a sequence as defined in SEQ ID NO:1, 2 or 3;
      c) a polynucleotide having a fragment of at least 50 consecutive nucleotides, or at least 100 consecutive nucleotides, or at least 200 consecutive nucleotides of a polynucleotide having a sequence as defined in SEQ ID NO:1, 2 or 3;
      d) a polynucleotide hybridizing under stringent conditions to a polynucleotide comprising at least 50 consecutive nucleotides, or at least 100 consecutive nucleotides, or at least 200 consecutive nucleotides of a polynucleotide having the sequence as defined in SEQ ID NO:1, 2 or 3; and
      e) a polynucleotide complementary to any of the polynucleotides of a) through d);
   2) and operably linked thereto one or more nucleic acids, wherein the operably linked nucleic acid encodes a polypeptide that is capable of conferring to a plant increased yield and/or increased stress tolerance to the plant;
B) selecting transgenic plants, wherein the plants have increased yield and/or increased stress tolerance, as compared to the wild type or null segregant plants.

Other embodiments of the invention relate to a nucleic acid construct or an expression vector comprising: a) a plant transcription regulatory element comprising a polynucleotide selected from the group consisting of: i) a polynucleotide having a sequence as defined in SEQ ID NO:1, 2 or 3; ii) a polynucleotide having 70% sequence identity to a polynucleotide having a sequence as defined in SEQ ID NO:1, 2 or 3; iii) a polynucleotide having a fragment of at least 50 consecutive nucleotides, or at least 100 consecutive nucleotides, or at least 200 consecutive nucleotides of a polynucleotide having a sequence as defined in SEQ ID NO:1, 2 or 3; iv) a polynucleotide hybridizing under stringent conditions to a polynucleotide comprising at least 50 consecutive nucleotides, or at least 100 consecutive nucleotides, or at least 200 consecutive nucleotides of a polynucleotide having the sequence as defined in SEQ ID NO:1, 2 or 3; and v) a polynucleotide complementary to any of the polynucleotides of a) through d); b) and operably linked thereto one or more nucleic acids.

An expression vector of the invention may comprise further regulatory elements. The term in this context is to be understood in a broad meaning, comprising all sequences which may influence construction or function of the expression vector. Regulatory elements may for example modify transcription and/or translation in prokaryotic or eukaryotic organism. The expression vector of the invention may comprise transcription regulatory element and—optionally additional regulatory elements—each operably liked to the nucleic acid to be expressed (or the transcription regulatory nucleotide).

A variety of 5′ and 3′ transcriptional regulatory sequences are available for use in the present invention. Transcriptional terminators are responsible for the termination of transcription and correct mRNA polyadenylation. The 3′ untranslated regulatory DNA sequence includes those from about 50 to about 1,000, or about 100 to about 1,000 nucleotide base pairs from the stop codon and contains plant transcriptional and translational termination sequences.

As the DNA sequence between the transcription initiation site and the start of the coding sequence, i.e., the untranslated leader sequence, can influence gene expression, one may also wish to employ a particular leader sequence. Leader sequences may include those predicted to direct optimum expression of the attached gene, i.e., to include a preferred consensus leader sequence, which may increase or maintain mRNA stability and prevent inappropriate initiation of translation. The choice of such sequences will be known to those of skilled in the art in light of the present disclosure. Sequences that are derived from genes that are highly expressed in plants will be preferred.

In another embodiment, regulatory elements also include the 5′-untranslated region, introns and the 3′-untranslated region of genes. Additional regulatory elements may include enhancer sequences or polyadenylation sequences.

An expression vector of the invention may comprise additional functional elements, which are to be understood in the broad sense as all elements that influence construction, propagation, or function of an expression vector or a transgenic organism comprising them. Such functional elements may include origin of replications (to allow replication in bacteria, for example, for the ORI of pBR322 or the P15A ori; Sambrook 1989), or elements required for Agrobacterium T-DNA transfer (such as for example the left and/or rights border of the T-DNA).

Additionally, the expression vector may be constructed and employed in the intracellular targeting of a specific gene product within the cells of a transgenic plant or in directing a protein to the extracellular environment. This will generally be achieved by joining a nucleic acid encoding a transit or signal peptide sequence to the coding sequence of a particular gene. The resultant transit or signal peptide will transport the protein to a particular intracellular or extracellular destination, respectively, and will then be post-translationally removed. Transit or signal peptides act by facilitating the transport of proteins through intracellular membranes, e.g., vacuole, vesicle, plastid and mitochondrial membranes, whereas signal peptides direct proteins through the extracellular membrane. By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. Targeting of certain proteins may be desirable in order to enhance the stability of the protein.

An expression vector may be utilized to insert a transcription regulatory nucleic acid of the invention into a plant genome. Such insertion will result in an operable linkage to a nucleic acid of interest, which as such already existed in the genome. By the insertion, the nucleic acid of interest is expressed in an endosperm-preferred way due to the transcription regulating properties of the transcription regulatory nucleotide. The insertion may be directed or by chance. Preferably the insertion is directed and realized by, for example, homologous recombination. By this procedure a natural promoter may be exchanged against the transcription regulatory nucleotide of the invention, thereby modifying the expression profile of an endogenous gene. The transcription regulatory nucleotide may also be inserted in a way, that antisense mRNA of an endogenous gene is expressed, thereby inducing gene silencing.

An operable linkage may, for example, comprise a sequential arrangement of the transcription regulatory nucleotide of the invention with a nucleic acid to be expressed, and—optionally—additional regulatory elements such as, for example, polyadenylation or transcription termination elements, enhancers, introns etc, can be included in a way that the transcription regulatory nucleotide can fulfill its function in the process of expressing the nucleic acid of interest under the appropriate conditions. The term “appropriate conditions” mean the presence of the expression vector in a plant cell. Preferred are arrangements, in which the nucleic acid of interest to be expressed is placed down-stream (i.e., in 3-direction) of the transcription regulatory nucleotide of the invention in a way that both sequences are covalently linked. Optionally, additional sequences may be inserted in-between the two sequences. Such sequences may be for example linker or multiple cloning sites. Furthermore, sequences can be inserted coding for parts of fusion proteins (in case a fusion protein of the protein encoded by the nucleic acid of interest is intended to be expressed). Preferably, the distance between the nucleic acid sequence of interest to be expressed and the transcription regulatory nucleotide of the invention is not more than 200 base pairs, or not more than 100 base pairs, or not more than 50 base pairs.

Plants may be transformed with the expression vector of the present invention by various methods known in the art. Any plant tissue capable of subsequent propagation may be transformed. The particular tissue chosen will vary depending on the propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and ultilane meristem).

Transformation of plants can be undertaken with a single DNA molecule or multiple DNA molecules (i.e., co-transformation), and both these techniques are suitable for use with the expression vector of the present invention. Numerous transformation vectors are available for plant transformation, and the expression vectors of this invention can be used in conjunction with any such vectors. The selection of vector will depend upon the preferred transformation technique and the target species for transformation. Expression vectors containing genomic DNA, cDNA or synthetic DNA fragments can be introduced into protoplasts or into intact tissues or isolated cells. Preferably expression vectors are introduced into intact tissues. General methods of culturing plant tissues are provided for example by Maki et al., (1993), and by Phillips et al. (1988). Preferably, expression vectors are introduced into maize or other plant tissues using a direct gene transfer method such as microprojectile-mediated delivery, DNA injection, electroporation and the like. More preferably expression vectors are introduced into plant tissues using the microprojectile media delivery with the biolistic device, for example, in Tomes et al. (1995). The vectors of the invention not only can be used for expression of structural genes but may also be used in exon-trap cloning, or promoter trap procedures to detect differential gene expression in varieties of tissues (Lindsey 1993; Auch & Reth 1990). Other transformation methods are available to those skilled in the art, such as direct uptake of foreign DNA constructs (EP 295959), techniques of electroporation (Fromm 1986) or high velocity ballistic bombardment with metal particles coated with the nucleic acid constructs (Kline 1987, and U.S. Pat. No. 4,945,050). Once transformed, the cells can be regenerated by those skilled in the art. It is particularly preferred to use the binary type vectors of Ti and Ri plasmids of Agrobacterium spp. Ti-derived vectors transform a wide variety of higher plants, including monocotyledonous and dicotyledonous plants, such as soybean, cotton, rape, tobacco, and rice (Pacciotti 1985: Byrne 1987; Sukhapinda 1987; Lorz 1985; Potrykus, 1985; Park 1985: Hiei 1994). The use of T-DNA to transform plant cells has received extensive study and is amply described (EP 120516; Hoekema, 1985; Knauf, 1983; and An 1985). For introduction into plants, the transcription regulatory elements of the invention can be inserted into binary vectors as described in the examples.

Transformation methods may include direct and indirect methods of transformation. Suitable direct methods include polyethylene glycol induced DNA uptake, liposome-mediated transformation (U.S. Pat. No. 4,536,475), biolistic methods using the gene gun (“particle bombardment”; Fromm M E et al. (1990) Bio/Technology. 8(9):833-9; Gordon-Kamm et al. (1990) Plant Cell 2:603), electroporation, incubation of dry embryos in DNA-comprising solution, and microinjection. In the case of these direct transformation methods, the plasmid used need not meet any particular requirements. Simple plasmids, such as those of the pUC series, pBR322, M13 mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch R B et al. (1985) Science 225:1229f. The Agrobacterium-mediated transformation is best suited for dicotyledonous plants but has also been adopted to monocotyledonous plants. The transformation of plants by Agrobacteria is described in White F F, Vectors for Gene Transfer in Higher Plants; Transgenic Plants, Vol. 1, Engineering and Utilization, edited by S. D. Kung and R. Wu, Academic Press, 1993, pp. 15-38; Jenes B et al. (1993) Techniques for Gene Transfer; Transgenic Plants, Vol. 1, Engineering and Utilization, edited by S. D. Kung and R. Wu, Academic Press, pp. 128-143; and Potrykus (1991) Annu Rev Plant Physiol Plant Molec Biol 42:205-225.

Transformation may result in transient or stable transformation and expression. Although a nucleotide of the present invention can be inserted into any plant and plant cell falling within these broad classes, it is particularly useful in crop plant cells.

Various tissues are suitable as starting material (explant) for the Agrobacterium-mediated transformation process including but not limited to callus (U.S. Pat. No. 5,591,616; EP-A1 604 662), immature embryos (EP-A1 672 752), pollen (U.S. Pat. No. 54,929,300), shoot apex (U.S. Pat. No. 5,164,310), or in planta transformation (U.S. Pat. No. 5,994,624). The method and material described herein can be combined with virtually all Agrobacterium mediated transformation methods known in the art. Preferred combinations include—but are not limited—to the following starting materials and methods:

| Variety | Material/Citation |
|---|---|
| Monocotyledonous | Immature embryos (EP-A1 672 752) |
| plants: | Callus (EP-A1 604 662) |
| | Embryogenic callus (U.S. Pat. No. 6,074,877) |
| | Inflorescence (U.S. Pat. No. 6,037,522) |
| | Flower (in planta) (WO 01/12828) |
| Banana | U.S. Pat. No. 5,792,935; EP-A1 731 632; U.S. Pat. No. 6,133,035 |
| Barley | WO 99/04618 |
| Maize | U.S. Pat. No. 5,177,010; U.S. Pat. No. 5,987,840 |
| Pineapple | U.S. Pat. No. 5,952,543; WO 01/33943 |
| Rice | EP-A1 897 013; U.S. Pat. No. 6,215,051; WO 01/12828 |
| Wheat | AU-B 738 153; EP-A1 856 060 |
| Beans | U.S. Pat. No. 5,169,770; EP-A1 397 687 |
| Brassica | U.S. Pat. No. 5,188,958; EP-A1 270 615; EP-A1 1,009,845 |
| Cacao | U.S. Pat. No. 6,150,587 |
| Citrus | U.S. Pat. No. 6,103,955 |
| Coffee | AU 729 635 |
| Cotton | U.S. Pat. No. 5,004,863; EP-A1 270 355; U.S. Pat. No. 5,846,797; EP-A1 1,183,377; |
| | EP-A1 1,050,334; EP-A1 1,197,579; EP-A1 1,159,436 |
| | Pollen transformation (U.S. Pat. No. 5,929,300) |
| | In planta transformation (U.S. Pat. No. 5,994,624) |
| Pea | U.S. Pat. No. 5,286,635 |
| Pepper | U.S. Pat. No. 5,262,316 |
| Poplar | U.S. Pat. No. 4,795,855 |
| Soybean | cotyledonary node of germinated soybean seedlings |
| | shoot apex (U.S. Pat. No. 5,164,310) |
| | axillary meristematic tissue of primary, or higher leaf node of |
| | about 7 days germinated soybean seedlings |
| | organogenic callus cultures |
| | dehydrated embryo axes |
| | U.S. Pat. No. 5,376,543; EP-A1 397 687; U.S. Pat. No. 5,416,011; U.S. Pat. No. 5,968,830; U.S. Pat. No. |
| | 5,563,055; U.S. Pat. No. 5,959,179; EP-A1 652 965; EP-A1 1,141,346 |
| Sugarbeet | EP-A1 517 833; WO 01/42480 |
| Tomato | U.S. Pat. No. 5,565,347 |

The nucleotides of the present invention can be directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit high expression levels. In one embodiment, the nucleotides are inserted into a plastid targeting vector and transformed into the plastid genome of a desired plant host. Plants homoplasmic for plastid genomes containing the nucleotide sequences are obtained, and are preferentially capable of high expression of the nucleotides.

Plastid transformation technology is for example extensively described in U.S. Pat. Nos. 5,451,513, 5,545,817, 5,545,818, and 5,877,462, in PCT application Nos. WO 95/16783 and WO 97/32977, and in McBride et al. (1994) Proc. Natl. Acad. Sci. USA 91, 7301-7305, all incorporated herein by reference in their entirety. The basic technique for plastid transformation involves introducing regions of cloned plastid DNA flanking a selectable marker together with the nucleotide sequence into a suitable target tissue, e.g., using biolistic or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al. (1990) Proc. Natl. Acad. Sci. USA 87, 8526-8530; Staub et al. (1992) Plant Cell 4, 39-45). The presence of cloning sites between these markers allows creation of a plastid targeting vector for introduction of foreign genes (Staub et al. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab et al. (1993) Proc. Natl. Acad. Sc. USA 90, 913-917). Other selectable markers useful for plastid transformation are known in the art and encompassed within the scope of the invention.

All of the compositions and methods disclosed and claimed herein can be made and executed without undue experimentation in light of the present disclosure. While the compositions and methods of this invention have been described in terms of certain embodiments, it will be apparent to those of skilled in the art that variations may be applied to the composition, methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. Throughout this application, various publications are referenced. The disclosures of all of these publications and those references cited within those publications in their entireties are hereby incorporated by reference into this application in order to more fully describe the state of the art to which this invention pertains.

The following examples are not intended to limit the scope of the claims to the invention, but are rather intended to be exemplary of certain embodiments. Any variations in the exemplified methods that occur to the skilled artisan are intended to fall within the scope of the present invention.

### EXAMPLES

### Materials and General Methods

Unless indicated otherwise, chemicals and reagents in the Examples were obtained from Sigma Chemical Company (St. Louis, Mo.), restriction endonucleases were from New England Biolabs (Beverly, Mass.) or Roche (Indianapolis, Ind.), oligonucleotides were synthesized by MWG Biotech Inc. (High Point, N.C.), and other modifying enzymes or kits regarding biochemicals and molecular biological assays were from Clontech (Palo Alto, Calif.), Pharmacia Biotech (Piscataway, N.J.), Promega Corporation (Madison, Wis.), or Stratagene (La Jolla, Calif.). Materials for cell culture media were obtained from Gibco/BRL (Gaithersburg, Md.) or DIFCO (Detroit, Mich.). The cloning steps carried out for the purposes of the present invention, such as, for example, restriction cleavages, agarose gel electrophoresis, purification of DNA fragments, transfer of nucleic acids to nitrocellulose and nylon membranes, linking DNA fragments, transformation of E. coli cells, growing bacteria, multiplying phages and sequence analysis of recombinant DNA, are carried out as described by Sambrook (1989). The sequencing of recombinant DNA molecules is carried out using ABI laser fluorescence DNA sequencer following the method of Sanger (Sanger 1977).

### Example 1

### Cloning of Maize Starch Synthase I Promoter and Rice Starch Synthase I Promoters and Terminators

The Zea mays Starch Synthase I cDNA (Knight et. al., The Plant Journal 1998) was used to perform a BLAST search against version 3 of the partially assembled Maize genome database maintained by The Institute for Genomic Research (Rockville, Md.). TIGR sequence AZM3_{—}58750 contains 1114 base pairs upstream of the start codon of the Starch Synthase I (SSI) cDNA (Genbank accession #AF036891). Primers were synthesized based on this sequence to amplify the unknown region further upstream. Polynucleotides from the tertiary round of thermal asymmetric interlaced PCR (TAIL PCR) (Liu et al., Plant J 1995 September; 8(3):457-463) using the primer pair EXS653 and EXS889 and genomic DNA from Maize line W64A were subsequently cloned and sequenced. A new primer pair, EXS890 and EXS918, were used to amplify and clone the proximal promoter region, including the 5′ UTR, resulting in a polynucleotide of 1887 base pairs (SEQ ID NO:1).

TABLE I
Primers used to clone the 5′ flanking sequence of Maize SSI.

| | | |
|---|---|---|
| Primer name | Sequence | Purpose |

| | | |
|---|---|---|
| EXS651 | NTC GAS TWT SGW GTT* | Arbitrary primer-Primary TAIL |
| (SEQ ID NO: 6) | | reaction |
| EXS652 | NGT CGA SWG ANA WGA A* | Arbitrary primer-Secondary TAIL |
| (SEQ ID NO: 7) | | reaction |
| EXS653 | WGT GNA GWA NCA NAG A* | Arbitrary primer-Tertiary TAIL |
| (SEQ ID NO: 8) | | reaction |
| EXS889 | GCCGTGTAGAACATGTCTACGATACCT | Reverse primer complementary to |
| (SEQ ID NO: 9) | | clone AZM3_58750 |
| EXS890 | TTTCATATGTGCGGAGAGGGAGAGCAG | Maize SSI promoter-Reverse |
| (SEQ ID NO: 10) | ACA | with start codon and NdeI |
| | | restriction site |
| EXS918 | TTTAAGCTTGTT TCATAAATGCTT | SSI promoter-Forward-1887 |
| (SEQ ID NO: 11) | TTCCTG ATTCCC T | relative to start codon. With |
| | | HindIII |

IUB codes for the degenerate bases used in the oligos:
N: G or A or T or C
S: C or G
W: A or T

Primer pairs were designed to amplify the proximal promoter region, including 5′ UTR, of the rice Starch Synthase I gene with Genbank accession # AB026295. The nested PCR strategy, using rice cv. Nipponbare genomic DNA as template, yielded a 2338 base pair amplicon (SEQ ID NO:3) that was subsequently cloned, resulting in the plasmid pEXS292.

TABLE 2
Primers used to clone the 5′ region of Rice SSI

| | | |
|---|---|---|
| EXS880 | CCGTCGCCATGgATCCCCCCTCCTC | Rice SSI promoter reverse w/ ATG |
| (SEQ ID NO: 12) | T | start codon and NcoI (Lower case G |
| | | is an added base to make NcoI) |
| EXS881 | CCAAGCTTGTAAATTTACACTAGCA | Rice SSI promoter forward starts at |
| (SEQ ID NO: 13) | AAATGCCCGT | −2338 relative to ATG start and has |
| | | HindIII |
| EXS883 | AACCATGgATCCCCCCTCCTCTCCG | Rice SSI promoter reverse w/ ATG |
| (SEQ ID NO: 14) | CCGATC | start codon and NcoI (Lower case G |
| | | is an added base to make NcoI) |

The rice SSI terminator region was PCR amplified from genomic DNA using standard techniques based on the known SSI genomic sequence (Genbank accession #AB026295; P0681F10.13). Two terminator fragments were cloned. Both contain the 3′ UTR sequence and either 300 (t-OSSSI-3, SEQ ID NO:4) or 500 bp (t-OSSSI-5, SEQ ID NO:5) downstream of the polyadenylation site. The plasmids are named 1000/SSITerm300 and 1000/SSITerm500, respectively.

TABLE 3
Primers used to amplify the
rice 3′ UTR and terminator regions

| | | |
|---|---|---|
| SSI Term300 KspI | ATTACCGCGGTAA | Forward primer common |
| (SEQ ID NO: 15) | ATGGATTTGAAGG | to both constructs |
| | AAGC | |
| SSI Term300 | ATATGGATCCCTT | Reverse primer |
| BamHI | CTCTATGCCATTA | |
| (SEQ ID NO: 16) | GGCC | |
| SSI Term500 | ATAGGATCCTGTC | Reverse primer |
| BamHI | AAATACCTTATAT | |
| (SEQ ID NO: 17) | TGC | |

### Example 2

### Vector Construction

Constructs were made using either the rice or maize SSI promoter plus GUS with either the Nos terminator (An G. at al., The Plant Cell 3:225-233, 1990) or rice SSI terminators (t-OSSSI) (Table 4).

TABLE 4
GUS chimeric constructs

| Binary | Composition of the expression cassette |
|---|---|
| vector | (promoter::reporter gene::terminator) |
| EXS1031 | Rice SSI promoter::GUS::Nos terminator |
| EXS1032 | Maize SSI (mutNcolNdel) promoter::GUS::Nos terminator |
| EXS1033 | Maize SSI promoter::GUS::Nos terminator |
| RLM661 | Rice SSI promoter::GUS::t-OSSSI-3 terminator |
| RLM62 | Rice SSI promoter::GUS::t-OSSSI-5 terminator |

### Example 3

### Maize Transformation

Agrobacterium cells harboring a plasmid containing the gene of interest and the maize AHAS gene were grown in YP medium supplemented with appropriate antibiotics for 1-2 days. Two loops of Agrobacterium cells were collected and suspended in 2 ml M-LS-002 medium (LS-inf. The cultures were incubated with shaking at 1,200 rpm for 5 min-3 hrs. Corncobs [genotype J553] were harvested at 8-11 days after pollination. The cobs were sterilized in 20% Clorox solution for 5 min, followed by spraying with 70% Ethanol and then thoroughly rinsing with sterile water. Immature embryos 0.8-2.0 mm in size were dissected into the tube containing Agrobacterium cells in LS-inf solution.

Agrobacterium infection of the embryos was carried out by inverting the tube several times. The mixture was poured onto a filter paper disk on the surface of a plate containing co-cultivation medium (M-LS-011). The liquid agro-solution was removed and the embryos were checked under a microscope and placed scutellum side up. Embryos were cultured in the dark at 22° C. for 2-4 days, and were transferred to M-MS-101 medium without selection and incubated for four to five days. Embryos were then transferred to M-LS-202 medium containing 0.75 μM imazethapyr and grown for four weeks to select for transformed callus cells.

Plant regeneration was initiated by transferring resistant calli to M-LS-504 medium supplemented with 0.75 μM imazethapyr and grown under light at 26° C. for two to three weeks. Regenerated shoots were then transferred to a rooting box with M-MS-618 medium (0.5 μM imazethapyr). Plantlets with roots were transferred to soil-less potting mixture and grown in a growth chamber for a week, then transplanted to larger pots and maintained in a greenhouse until maturity.

### Example 4

### Endosperm-Preferred Expression in Maize

The transgenic lines transformed with construct EXS1031, EXS1032, EXS1033, RLM661 and RLM662 containing the maize or rice SSI promoter were grown and the indicated tissues and organs were assayed for GUS activity at the indicated time period by staining. Expression begins between 5 and 10 DAP (days after pollination) with high levels of endosperm specific expression occurring by 15 DAP and continuing through maturity. Expression of GUS is not found in other tissues or organs tested.

The following scoring index was used: “−” for no staining, “+” for weak staining, “++” for strong staining, “+++” for very strong staining.

TABLE 5
GUS activity as assayed by β-Glucuronidase staining in
EXS1031, EXS1032 and EXS1033 lines.

| Tissue | | GUS Activity |
|---|---|---|
| Roots and leaves at 5-leaf stage (~3 weeks after | | − |
| germination) | | |
| Leaves at flowering stage (first emergence of silk) | | − |
| Spikelets/Tassel (at pollination) | | − |
| Stem | | − |
| Kernel tissue (DAP) | | |
| Endosperm | 5 | − |
| Embryo | 5 | − |
| Endosperm | 10 | + |
| Embryo | 10 | + |
| Endosperm | 15 | ++ |
| Embryo | 15 | + |
| Endosperm | 20 | +++ |
| Embryo | 20 | + |
| Endosperm | 25 | +++ |
| Embryo | 25 | + |
| Endosperm | 30 | +++ |
| Embryo | 30 | + |

The staining patterns observed in maize lines carrying GUS under the control of the rice SSI promoter (EXS1031) and mutated maize SSI (EXS1032) were nearly identical to the staining pattern of plants carrying the maize SSI promoter (EXS1033).

Plants containing the rice SSI terminator regions (RLM661 and RLM662) were grown to maturity and dried kernels were assayed for GUS expression by staining as above. GUS expression patterns in kernels of these plants were similar to those carrying the Nos terminator confirming efficient termination of transcription of the reporter gene by the rice SSI terminator. Both the 300 and 500 bp terminator polynucleotides showed identical staining patterns.

### Example 5

### Utilization of Transgenic Crops

A reporter gene in EXS1031, EXS1032, EXS1033, RLM661 or RLM662 can be replaced with a gene of interest to express in an endosperm-preferred manner and confer agronomically desired trait to a plant, for example, increased nutritional value, increase yield, increased stress tolerance, or increased or altered starch content, and the like. The expression cassettes are transformed into monocotyledonous plants. Standard methods for transformation in the art can be used if required. Transformed plants are regenerated using known methods. Various phenotypes are measured to determine improvement of amino acid composition, oil quality and quantity, starch quality and quantity, yield, stress tolerance under, for example, drought conditions. Gene expression levels are determined at different stages of development and at different generations (T0 to T2 plants or further generations). Results of the evaluation in plants determines appropriate genes in combination with the promoters to increase nutritional value, increased or modified starch content, increased yield, increased stress tolerance, and the like.

1. An isolated plant transcription regulatory element comprising a polynucleotide selected from the group consisting of:
a) a polynucleotide having a sequence as defined in SEQ ID NO: 1, 2, or 3;
b) a polynucleotide having 70% sequence identity to a polynucleotide having a sequence as defined in SEQ ID NO:1, 2, or 3;
c) a polynucleotide having a fragment of at least 50 consecutive nucleotides, or at least 100 consecutive nucleotides, or at least 200 consecutive nucleotides of a polynucleotide having a sequence as defined in SEQ ID NO: 1, 2, or 3;
d) a polynucleotide hybridizing under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:1, 2, or 3; and
e) a polynucleotide complementary to any of the polynucleotides of a) through d).

2. The isolated plant transcription regulatory element of claim 1, wherein the plant transcription regulatory element regulates endosperm-preferred expression of a gene of interest in a plant or plant cell.

3. The isolated plant transcription regulatory element of claim 1, wherein the plant transcription regulatory element is operably linked to one or more heterologous nucleic acids.

4. The isolated plant transcription regulatory element of claim 1, wherein the nucleic acid has a sequence as defined in SEQ ID NO: 1, 2, or 3.

5. The isolated plant transcription regulatory element of claim 1, wherein the polynucleotide has 70% sequence identity to a polynucleotide having a sequence as defined in SEQ ID NO:1, 2, or 3.

6. The isolated plant transcription regulatory element of claim 1, wherein the polynucleotide comprises a fragment of at least 50 consecutive nucleotides, or at least 100 consecutive nucleotides, or at least 200 consecutive nucleotides of a polynucleotide having a sequence as defined in SEQ ID NO: 1, 2, or 3.

7. The isolated plant transcription regulatory element of claim 1, wherein the polynucleotide hybridizes under stringent conditions to a polynucleotide comprising at least 50 consecutive nucleotides, or at least 100 consecutive nucleotides, or at least 200 consecutive nucleotides of a polynucleotide having a sequence as defined in SEQ ID NO: 1, 2, or 3.

8. An isolated plant transcription terminator element comprising a polynucleotide selected from the group consisting of:
a) a polynucleotide having a sequence as defined in SEQ ID NO.:4 or SEQ ID NO:5;
b) a polynucleotide having 70% sequence identity to a polynucleotide having a sequence as defined in SEQ ID NO:4 or SEQ ID NO:5;
c) a polynucleotide having a fragment of at least 50 consecutive nucleotides, or at least 100 consecutive nucleotides, or at least 200 consecutive nucleotides of a polynucleotide having a sequence as defined in SEQ ID NO:4 or SEQ ID NO:5;
d) a polynucleotide hybridizing under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:4 or SEQ ID NO:5; and
e) a polynucleotide complementary to any of the polynucleotides of a) through d).

9. A plant transformed with an isolated plant transcription regulatory element comprising a polynucleotide selected from the group consisting of:
a) a polynucleotide having a sequence as defined in SEQ ID NO: 1, 2, or 3;
b) a polynucleotide having 70% sequence identity to a polynucleotide having a sequence as defined in SEQ ID NO: 1, 2, or 3;
c) a polynucleotide having a fragment of at least 50 consecutive nucleotides, or at least 100 consecutive nucleotides, or at least 200 consecutive nucleotides of a polynucleotide having a sequence as defined in SEQ ID NO: 1, 2, or 3;
d) a polynucleotide hybridizing under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:1, 2, or 3; and
e) a polynucleotide complementary to any of the polynucleotides of a) through d).

10. The plant transformed with an isolated plant transcription regulatory element of claim 9, wherein the plant transcription regulatory element regulates expression of one or more operably linked heterologous nucleic acids in a plant or plant cell.

11. The plant of claim 9, wherein the plant is monocotyledonous or dicotyledonous.

12. The plant of claim 9, wherein the expression of the transcription regulatory element is endosperm-preferred expression.

13. The plant of claim 9, wherein the polynucleotide has a sequence as defined in SEQ ID NO:1, 2, or 3.

14. The plant of claim 9, wherein the polynucleotide has 70% sequence identity to a polynucleotide having a sequence as defined in SEQ ID NO: 1, 2, or 3.

15. The plant of claim 9, wherein the polynucleotide has a fragment of at least 50 consecutive nucleotides, or at least 100 consecutive nucleotides, or at least 200 consecutive nucleotides of a polynucleotide having a sequence as defined in SEQ ID NO: 1, 2, or 3.

16. The plant of claim 9, wherein the polynucleotide hybridizes under stringent conditions to a polynucleotide comprising at least 50 consecutive nucleotides, or at least 100 consecutive nucleotides, or at least 200 consecutive nucleotides of a polynucleotide having a sequence as defined in SEQ ID NO:1, 2, or 3.

17. A plant seed produced by the plant of claim 9, wherein the seed comprises the isolated plant transcription regulatory element.

18. The plant of claim 10, wherein said operably linked heterologous nucleic acid encodes a polypeptide that confers to a plant a trait or property selected from increased yield, increased resistance under stress conditions, increased nutritional quality, increased or modified starch content, or increased or modified oil content of a seed or sprout.

19. The plant of claim 10, wherein said operably linked heterologous nucleic acid expresses regulatory RNA selected from the group consisting of dsRNA, siRNA and miRNA.

20. A nucleic acid construct comprising the plant transcription regulatory element polynucleotide of claim 1 operably linked to one or more heterologous nucleic acids.

21. An expression vector comprising the nucleic acid construct of claim 20.

22. A plant cell having stably incorporated into its genome the nucleic construct of claim 20.

23. A method for conferring increased yield, increased stress tolerance, increased nutritional quality, increased nutritional value, increased or modified starch content, or increased or modified oil content of a seed or a sprout to a plant, wherein the method comprises the steps of:
a) introducing into a plant cell or a plant the expression vector of claim 21, wherein the operably linked nucleic acid encodes a polypeptide or RNA that is capable of conferring to a plant increased yield, increased stress tolerance, increased nutritional quality, increased nutritional value, increased or modified starch, or increased or modified oil content to the plant; and
b) selecting transgenic plants, wherein the plants have increased yield, increased stress tolerance under stress conditions, increased nutritional quality, increased nutritional value, increased or modified starch content, or increased or modified oil content of a seed or a sprout of the plants, as compared to the wild type or null segregant plants.

24. The method of claim 23, wherein the plant transcription regulatory element regulates tissue-preferred expression of the functionally linked heterologous nucleic acid in a plant or plant cell.

25. A plant seed produced by the plait of claim 10, wherein the seed comprises the isolated plant transcription regulatory element.
